(19) United States
(12) Patent Application Publication
(10) Pub. No.: US 2012/0146264 A1
(43) Pub. Date: Jun. 14, 2012

(54) RESIN COMPOSITION FOR LASER ENGRAVING, RELIEF PRINTING PLATE PRECURSOR FOR LASER ENGRAVING AND PROCESS FOR PRODUCING SAME, AND PROCESS FOR MAKING RELIEF PRINTING PLATE

(75) Inventor: Takashi KAWASHIMA, Haibara-gun (JP)

(73) Assignee: FUJIFILM CORPORATION, Tokyo (JP)

(21) Appl. No.: 13/316,148

(22) Filed: Dec. 9, 2011

(30) Foreign Application Priority Data
Dec. 10, 2010 (JP) 2010-275997

(51) Int. Cl. B29C 59/16; C08F 228/02; C08F 230/08; C08L 41/00; C08L 35/00; C07F 7/18; C07D 209/76; C07D 405/06; C07C 311/39; C07D 207/448; C07D 403/10; C08F 222/40; C07C 271/20

(52) U.S. Cl. 264/400; 526/262; 526/288; 526/279; 525/55; 524/547; 524/548; 523/102; 525/326.8; 525/328.5; 560/22; 548/435; 548/465; 564/86; 548/549; 548/524; 548/406

(57) ABSTRACT

A resin composition for laser engraving is provided that includes a low molecular weight compound containing at least one type of polymerizable group selected from the group consisting of an ethylenically unsaturated group, an epoxy group, an oxetanyl group, a hydrolyzable silyl group, and a silanol group and further containing a sulfonamide group, a low molecular weight compound containing a residue selected from the group consisting of a maleimide group, a succinimide group, and a phthalimide group, or a polymer compound containing a constituent unit derived from a compound containing a maleimide group or a constituent unit containing a sulfonamide group.

### TECHNICAL FIELD

The present invention relates to a resin composition for laser engraving, a relief printing plate precursor for laser engraving and a process for producing same, and a process for making a relief printing plate.

### BACKGROUND ART

As a process for forming a printing plate by forming asperities in a photosensitive resin layer layered on a support surface area, a method in which a relief-forming layer formed using a photosensitive composition is exposed to UV light through an original image film to thus selectively cure an image area, and an uncured area is removed using a developer, the so-called ‘analogue plate making’, is well known.

A relief printing plate is a letterpress printing plate having a relief layer with asperities, and such a relief layer with asperities is obtained by patterning a relief-forming layer comprising a photosensitive composition containing as a main component, for example, an elastomeric polymer such as a synthetic rubber, a resin such as a thermoplastic resin, or a mixture of a resin and a plasticizer, thus forming asperities. Among such relief printing plates, one having a soft relief layer is sometimes called a flexographic plate.

When a relief printing plate is made by analogue plate making, since an original image film employing a silver salt material is generally necessary, production time and cost for the original image film are incurred. Furthermore, since development of the original image film requires a chemical treatment, and treatment of development effluent is required, simpler plate making methods, for example, a method that does not use an original image film, a method that does not require development processing, etc. have been examined.

In recent years, methods for carrying out plate-making of a relief-forming layer by scanning exposure without requiring an original image film have been investigated.

As a technique that does not require an original image film, a relief printing plate precursor in which a laser-sensitive mask layer element that can form an image mask is provided above a relief-forming layer has been proposed (ref. e.g. JP-A-2004-262077 (JP-A denotes a Japanese unexamined patent application publication)). Such a process for making a precursor is called a ‘mask CTP method’ because an image mask having a similar function to that of an original image film is formed from a mask layer element by irradiation with a laser based on image data, but although no original image film is required, the subsequent plate-making process is a step of removing an uncured part by development involving exposure with UV light via an image mask, and there is still room for improvement in terms of development processing still being required.

As a plate making process that does not require a development process, many of the so-called ‘direct engraving CTP methods’, in which a relief-forming layer is directly engraved by means of a laser, have been proposed. The direct engraving CTP method is a method in which relief-forming asperities are formed by engraving by means of the laser itself, and has the advantage that, unlike relief formation using an original image film, the relief shape can be freely controlled.

With regard to conventional plate materials that have been used in the direct engraving CTP method, a large number of proposals have been made, such as one in which, as a binder that determines the properties of the plate material, a hydrophobic elastomer (rubber) is used (e.g. JP-A-2004-262077).

### SUMMARY OF THE INVENTION

### Problems to be Solved by the Invention

A relief plate obtained by laser-engraving a relief-forming layer is required to have resistance toward a so-called UV ink, which is UV-curable, resistance toward an aqueous ink, and resistance toward a washing liquid.

It is an object of the present invention to provide a resin composition for laser engraving that is suitable for a relief-forming layer of a relief printing plate precursor for laser engraving, the relief-forming layer having excellent resistance toward a rinsing liquid for engraving residue formed by laser engraving, high resistance toward an aqueous ink during printing, and excellent resistance toward a UV ink.

Furthermore, it is another object of the present invention to provide a relief printing plate precursor for laser engraving, the relief layer formed having excellent resistance toward an ink washing liquid, resistance toward an aqueous ink, and resistance toward a UV ink, and to provide a process for producing the precursor, and a process for producing a relief printing plate, the process employing the precursor.

### Means for Solving the Problems

The above-mentioned objects have been accomplished by means <1>, <9> to <11>, and <13> below. They are listed below together with <2> to <8>, <12>, <14>, and <15>, which are preferred embodiments.

<1> A resin composition for laser engraving, comprising (Component A) a low molecular weight compound containing at least one type of polymerizable group selected from the group consisting of an ethylenically unsaturated group, an epoxy group, an oxetanyl group, a hydrolyzable silyl group, and a silanol group and further containing a sulfonamide group, a low molecular weight compound containing a residue selected from the group consisting of a maleimide group, a succinimide group and a phthalimide group, or a polymer compound containing a constituent unit derived from a compound containing a maleimide group or a constituent unit containing a sulfonamide group,
<2> the resin composition for laser engraving according to <1>, wherein the polymer compound contains an ethylenically unsaturated group or a hydrolyzable silyl group,
<3> the resin composition for laser engraving according to <1> or <2>, wherein it further comprises (Component B) a binder polymer,
<4> the resin composition for laser engraving according to any one of <1> to <3>, wherein it further comprises (Component C) a polymerizable compound,
<5> the resin composition for laser engraving according to any one of <1> to <4>, wherein it further comprises (Component D) a polymerization initiator,
<6> the resin composition for laser engraving according to any one of <1> to <5>, wherein it further comprises (Component E) a plasticizer,
<7> the resin composition for laser engraving according to any one of <1> to <6>, wherein it further comprises (Component F) a fragrance,
<8> the resin composition for laser engraving according to any one of <1> to <7>, wherein it further comprises (Component G) a photothermal conversion agent that can absorb light having a wavelength of 700 to 1,300 nm,
<9> a relief printing plate precursor for laser engraving comprising above a support a relief-forming layer comprising the resin composition for laser engraving according to any one of <1> to <8>,
<10> a relief printing plate precursor for laser engraving comprising above a support a relief-forming layer formed by crosslinking a relief-forming layer comprising the resin composition for laser engraving according to any one of <1> to <8> by light and/or heat,
<11> a process for producing a relief printing plate precursor, comprising a layer formation step of forming a relief-forming layer from the resin composition for laser engraving according to any one of <1> to <8>, and a crosslinking step of forming a crosslinked relief-forming layer by crosslinking the relief-forming layer by light and/or heat,
<12> the process for producing a relief printing plate precursor according to <11>, wherein the crosslinking step is a step of crosslinking the relief-forming layer by heat,
<13> a process for making a relief printing plate, comprising a relief layer formation step of forming a relief layer by laser-engraving a relief-forming layer of a relief printing plate precursor produced by the process for producing a relief printing plate precursor according to <11> or <12>,
<14> the process for making a relief printing plate according to <13>, wherein the relief-forming layer has a thickness of at least 0.05 mm but no greater than 10 mm, and
<15> the process for making a relief printing plate according to <13> or <14>, wherein the relief-forming layer has a Shore A hardness of at least 50° but no greater than 90°.

### MODE FOR CARRYING OUT THE INVENTION

The present invention is explained in detail below.

### Resin Composition for Laser Engraving

The resin composition for laser engraving of the present invention comprises (Component A) a low molecular weight compound containing at least one type of polymerizable group selected from the group consisting of an ethylenically unsaturated group, an epoxy group, an oxetanyl group, a hydrolyzable silyl group, and a silanol group and further containing a sulfonamide group, a low molecular weight compound containing a residue selected from the group consisting of a maleimide group, a succinimide group, and a phthalimide group, or a polymer compound containing a constituent unit derived from a compound containing a maleimide group or a constituent unit containing a sulfonamide group.

Hereinafter, a ‘constituent unit derived from a compound containing a maleimide group’ may simply be called a ‘constituent unit derived from a maleimide group’.

In addition to the application to the relief-forming layer of the relief printing plate precursor, to which the laser engraving is to be given, the resin composition for laser engraving of the present invention can be widely applied to other applications. For example, the composition can be applied not only to the relief-forming layer of a printing plate precursor in which a convex relief is formed by laser engraving to be described in detail below, but also to other material in which asperities or apertures are formed on the surface, for example, to the formation of various printing plates and various formed bodies in which images are formed by laser engraving such as an intaglio plate, a stencil plate, and a stamp.

Among them, the application of this composition to the relief-forming layer disposed over an appropriate support is preferable.

In the present specification, with regard to explanation of a relief printing plate precursor, a layer having a flat surface as an image formation layer, preferably comprising a binder polymer, and being subjected to laser engraving is called a relief-forming layer, and a layer having asperities on the surface formed by laser engraving the above layer is called a relief layer.

As described above, Component A is selected from the group consisting of two types of low molecular weight compounds (Component A1) and (Component A2) and one type of polymer compound (Component A3). These compounds are explained below.

(Component A1) Low Molecular Weight Compound Containing at Least One Type of Polymerizable Group Selected from the Group Consisting of Ethylenically Unsaturated Group, Epoxy Group, Oxetanyl Group, Hydrolyzable Silyl Group, and Silanol Group and Further Containing Sulfonamide Group

Here, the ethylenically unsaturated group, the epoxy group, and the oxetanyl group, the hydrolyzable silyl group, and the silanol group all have self-reactivity or addition reactivity and common properties in terms of the capability of changing into a higher molecular weight compound by a chain-addition reaction or bonding to another active crosslinkable group. Component A1 is a low molecular weight compound containing at least one polymerizable group selected from the above-mentioned groups and further containing a sulfonamide group.

Here, the ‘low molecular weight compound’ means the molecular weight being less than 1,000, and the molecular weight is preferably 100 to 900. Furthermore, the ‘polymer compound’ as Component A3, which is described later, means the molecular weight being at least 1,000, and from the viewpoint of compatibility with other materials used, the weight-average molecular weight is preferably 1,000 to 1,000,000, and more preferably 1,000 to 100,000.

The ethylenically unsaturated group is limited to an open-chain structure; examples thereof include a (meth)acryloyl group, a vinyl group, and an allyl group, and a (meth)acryloyl group and a vinyl group are preferable.

The epoxy group may be open-chain or cyclic, and examples thereof include a glycidyl group and a cyclohexene oxide group.

The oxetanyl group may have a substituent, and is preferably unsubstituted or substituted with a lower alkyl group having 1 to 5 carbons.

The ‘hydrolyzable silyl group’ of the ‘hydrolyzable silyl group and silanol group’ is a silyl group that is hydrolyzable; examples of hydrolyzable groups include an alkoxy group, a mercapto group, a halogen atom, an amide group, an acetoxy group, an amino group, and an isopropenoxy group. A silyl group is hydrolyzed to become a silanol group, and a silanol group undergoes dehydration-condensation to form a siloxane bond. Such a hydrolyzable silyl group or silanol group is preferably one represented by Formula (1) below.

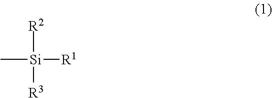

In Formula (1) above, R¹ to R³ denote independently a hydrolyzable group selected from the group consisting of an alkoxy group, an aryloxy group, a mercapto group, a halogen atom, an amide group, an acetoxy group, an amino group, and an isopropenoxy group, or a hydroxy group, a hydrogen atom, or a monovalent organic group. At least one of R¹ to R³ denotes a hydrolyzable group selected from the group consisting of an alkoxy group, an aryloxy group, a mercapto group, a halogen atom, an amide group, an acetoxy group, an amino group, and an isopropenoxy group, or a hydroxy group.

When R¹ to R³ denote a monovalent organic group, from the viewpoint that solubility in various types of organic solvents can be given, an organic group is preferably an alkyl group having 1 to 30 carbon atoms.

In Formula (1) above, the hydrolyzable group bonded to the silicon atom is particularly preferably an alkoxy group or a halogen atom.

From the viewpoint of rinsing properties and printing durability, the alkoxy group is preferably an alkoxy group having 1 to 30 carbon atoms, more preferably an alkoxy group having 1 to 15 carbon atoms, yet more preferably an alkoxy group having 1 to 5 carbon atoms, particularly preferably an alkoxy group having 1 to 3 carbon atoms.

Furthermore, examples of the halogen atom include a F atom, a Cl atom, a Br atom, and a I atom, and from the viewpoint of ease of synthesis and stability it is preferably a Cl atom or a Br atom, and more preferably a Cl atom.

‘A compound having a polymerizable group which is a hydrolyzable silyl group or a silanol group’ as Component A is preferably a compound having one or more groups represented by Formula (1) above, and more preferably a compound having two or more. A compound having two or more hydrolyzable silyl groups is particularly preferably used. That is, a compound having in the molecule two or more silicon atoms having a hydrolyzable group bonded thereto is preferably used. The number of silicon atoms having a hydrolyzable group bond thereto contained in the compound is preferably at least 2 but no greater than 6, and most preferably 2 or 3.

A range of 1 to 3 of the hydrolyzable groups may bond to one silicon atom, and the total number of hydrolyzable groups in Formula (1) is preferably in a range of 2 or 3. It is particularly preferable that three hydrolyzable groups are bonded to a silicon atom. When two or more hydrolyzable groups are bonded to a silicon atom, they may be identical to or different from each other.

Examples of the alkoxy group include a methoxy group, an ethoxy group, a propoxy group, an isopropoxy group, a butoxy group, a tert-butoxy group, and a benzyloxy group. Examples of the alkoxysilyl group having an alkoxy group bonded thereto include a trialkoxysilyl group such as a trimethoxysilyl group, a triethoxysilyl group, a triisopropoxysilyl group, or a triphenoxysilyl group; a dialkoxymonoalkylsilyl group such as a dimethoxymethylsilyl group or a diethoxymethylsilyl group; and a monoalkoxydialkylsilyl group such as a methoxydimethylsilyl group or an ethoxydimethylsilyl group. A plurality of each of these alkoxy groups may be used in combination, or a plurality of different alkoxy groups may be used in combination.

Examples of the aryloxy group include phenoxy group. Examples of the aryloxysilyl group having an aryloxy group bonded thereto include a triarylsilyl group such as a triphenylsilyl group.

The sulfonamide group in Component A1 is preferably unsubstituted, and is preferably a sulfonamide group that is substituted on a phenylene group.

As a specific example of Component A1, A-4, which is used in the Examples described later, can be cited.

(Component A2) Low Molecular Weight Compound Containing Residue Selected from the Group Consisting of Maleimide Group, Succinimide Group, and Phthalimide Group

Component A2 is a low molecular weight compound containing a maleimide group, a succinimide group, or a phthalimide group, which have the common feature of being cyclic residues. The ‘low molecular weight’ referred to here has the same meaning as that of the ‘low molecular weight’ in Component A1.

The succinimide group may be condensed with an alicyclic moiety.

Furthermore, with regard to the maleimide group, one or more groups may be present in one molecule. A compound containing two or more maleimide groups functions as a curable ethylenically unsaturated compound and is preferable as Component A2.

Specific examples of Component A2 include A-2, A-3, and A-5 to A-8 in specific examples of Component A used in the Examples.

(Component A3) Polymer Compound Containing Constituent Unit Derived from Maleimide Group or Constituent Unit Containing Sulfonamide Group

Component A3 is a polymer compound; as described above the ‘polymer compound’ means the molecular weight being at least 1,000, and the weight-average molecular weight is preferably 1,000 to 1,000,000, and more preferably 1,000 to 100,000.

As Component A, Component A3 is preferred over Component A1 and Component A2.

Component A3 is a polymer compound containing a constituent unit derived from a maleimide group or a constituent unit containing a sulfonamide group; from the viewpoint of achieving a balance between solubility and ink resistance/washing liquid resistance, the copolymerization ratio of the constituent unit containing a maleimide group or a sulfonamide group is preferably 5 to 80 mol % with the entire constituent units as 100 mol %, and more preferably 10 to 60 mol %. It preferably contains a constituent unit containing at least one type of polymerizable group selected from the group consisting of an ethylenically unsaturated group, an epoxy group, an oxetanyl group, a hydrolyzable silyl group, and a silanol group. It is also preferable for Component A3 to contain as another polymerizable group a constituent unit containing an isocyanato group.

The constituent unit constituting the polymer of Component A3 is derived from a compound containing an acyclic ethylenically unsaturated group, from a compound containing a hydrolyzable silyl group and an acyclic ethylenically unsaturated group, or from a compound containing an acyclic ethylenically unsaturated group and a p-sulfonamidophenyl group.

The content of Component A contained in the resin composition of the present invention is preferably in the range of 0.1 to 80 weight % on a solids content basis, more preferably in the range of 1 to 60 weight %, and most preferably in the range of 5 to 60 weight %.

Specific examples of component A are listed below.

Methacrylamides such as allyl[3-(2-nitrobenzenesulfonamido)propyl]carbamate, N-allyloxycarbonyl-2-nitrobenzenesulfonamide, N-(o-aminosulfonylphenyl)methacrylamide, N-(m-aminosulfonylphenyl)methacrylamide, N-(p-aminosulfonylphenyl)methacrylamide, N-(o-methylaminosulfonylphenyl)methacrylamide, N-(m-methylaminosulfonylphenyl)methacrylamide, N-(p-methylaminosulfonylphenyl)methacrylamide, N-(o-ethylaminosulfonylphenyl)methacrylamide, N-(m-ethylaminosulfonylphenyl)methacrylamide, N-(p-ethylaminosulfonylphenyl)methacrylamide, N-(o-n-propylaminosulfonylphenyl)methacrylamide, N-(m-n-propylaminosulfonylphenyl)methacrylamide, N-(p-n-propylaminosulfonylphenyl)methacrylamide, N-(o-i-propylaminosulfonylphenyl)methacrylamide, N-(m-i-propylaminosulfonylphenyl)methacrylamide, N-(p-i-propylaminosulfonylphenyl)methacrylamide, N-(o-n-butylaminosulfonylphenyl)methacrylamide, N-(m-n-butylaminosulfonylphenyl)methacrylamide, N-(p-n-butylaminosulfonylphenyl)methacrylamide, N-(o-i-butylaminosulfonylphenyl)methacrylamide, N-(m-i-butylaminosulfonylphenyl)methacrylamide, N-(p-i-butylaminosulfonylphenyl)methacrylamide, N-(o-sec-butylaminosulfonylphenyl)methacrylamide, N-(m-sec-butylaminosulfonylphenyl)methacrylamide, N-(p-sec-butylaminosulfonylphenyl)methacrylamide, N-(o-t-butylaminosulfonylphenyl)methacrylamide, N-(m-t-butylaminosulfonylphenyl)methacrylamide, N-(p-t-butylaminosulfonylphenyl)methacrylamide, N-(o-phenylaminosulfonylphenyl)methacrylamide, N-(m-phenylaminosulfonylphenyl)methacrylamide, N-(p-phenylaminosulfonylphenyl)methacrylamide, N-(o-(α-naphthylaminosulfonyl)phenyl)methacrylamide, N-(m-(α-naphthylaminosulfonyl)phenyl)methacrylamide, N-(p-(α-naphthylaminosulfonyl)phenyl)methacrylamide, N-(o-(β-naphthylaminosulfonyl)phenyl)methacrylamide, N-(m-(β-naphthylaminosulfonyl)phenyl)methacrylamide, N-(p-(βnaphthylaminosulfonyl)phenyl)methacrylamide, N-(1-(3-aminosulfonyl)naphthyl)methacrylamide, N-(1-(3-methylaminosulfonyl)naphthyl)methacrylamide, N-(1-(3-ethylaminosulfonyl)naphthyl)methacrylamide, N-(o-methylsulfonylaminophenyl)methacrylamide, N-(m-methylsulfonylaminophenyl)methacrylamide, N-(p-methylsulfonylaminophenyl)methacrylamide, N-(o-ethylsulfonylaminophenyl)methacrylamide, N-(m-ethylsulfonylaminophenyl)methacrylamide, N-(p-ethylsulfonylaminophenyl)methacrylamide, N-(o-phenylsulfonylaminophenyl)methacrylamide, N-(m-phenylsulfonylaminophenyl)methacrylamide, N-(p-phenylsulfonylaminophenyl)methacrylamide, N-(o-(p-methylphenylsulfonylamino)phenyl)methacrylamide, N-(m-(p-methylphenylsulfonylamino)phenyl)methacrylamide, N-(p-(p-methylphenylsulfonylamino)phenyl)methacrylamide, N-(p-(α-naphthylsulfonylamino)phenylmethacrylamide, N-(p-(β-naphthylsulfonylamino)phenyl)methacrylamide, N-(2-methylsulfonylaminoethyl)methacrylamide, N-(2-ethylsulfonylaminoethyl)methacrylamide, N-(2-phenylsulfonylaminoethyl)methacrylamide, N-(2-p-methylphenylsulfonylaminoethyl)methacrylamide, N-(2-α-naphthylsulfonylaminoethyl)methacrylamide, N-(2-β-naphthylsulfonylamino)ethylmethacrylamide, N-(m-dimethylaminosulfonylphenyl)methacrylamide, N-(p-dimethylaminosulfonylphenyl)methacrylamide, N-(o-diethylaminosulfonylphenyl)methacrylamide, N-(m-diethylaminosulfonylphenyl)methacrylamide, and N-(p-diethylaminosulfonylphenyl)methacrylamide, acrylamides having the same substituents as above,

methacrylic acid esters such as o-aminosulfonylphenyl methacrylate, m-aminosulfonylphenyl methacrylate, p-aminosulfonylphenyl methacrylate, o-methylaminosulfonylphenyl methacrylate, m-methylaminosulfonylphenyl methacrylate, p-methylaminosulfonylphenyl methacrylate, o-ethylaminosulfonylphenyl methacrylate, m-ethylaminosulfonylphenyl methacrylate, p-ethylaminosulfonylphenyl methacrylate, o-n-propylaminosulfonylphenyl methacrylate, m-n-propylaminosulfonylphenyl methacrylate, p-n-propylaminosulfonylphenyl methacrylate, o-i-propylaminosulfonylphenyl methacrylate, m-i-propylaminosulfonylphenyl methacrylate, o-n-butylaminosulfonylphenyl methacrylate, m-n-butylaminosulfonylphenyl methacrylate, p-n-butylaminosulfonylphenyl methacrylate, m-i-butylaminosulfonylphenyl methacrylate, p-i-butylaminosulfonylphenyl methacrylate, m-sec-butylaminosulfonylphenyl methacrylate, p-sec-butylaminosulfonylphenyl methacrylate, m-t-butylaminosulfonylphenyl methacrylate, p-t-butylaminosulfonylphenyl methacrylate, o-phenylaminosulfonylphenyl methacrylate, m-phenylaminosulfonylphenyl methacrylate, p-phenylaminosulfonylphenyl methacrylate, m-(α-naphthylaminosulfonyl)phenyl methacrylate, p-(α-naphthylaminosulfonylphenyl)methacrylate, m-(β-naphthylaminosulfonyl)phenyl methacrylate, p-(β-naphthylaminosulfonyl)phenyl methacrylate, 1-(3-aminosulfonyl)naphthyl methacrylate, 1-(3-methylaminosulfonyl)naphthyl methacrylate, 1-(3-ethylaminosulfonyl)naphthyl methacrylate, o-methylsulfonylaminophenyl methacrylate, m-methylsulfonylaminophenyl methacrylate, p-methylsulfonylaminophenyl methacrylate, o-ethylsulfonylaminophenyl methacrylate, m-ethylsulfonylaminophenyl methacrylate, p-ethylsulfonylaminophenyl methacrylate, o-phenylsulfonylaminophenyl methacrylate, m-phenylsulfonylaminophenyl methacrylate, p-phenylsulfonylaminophenyl methacrylate, o-(p-methylphenylsulfonylamino)phenyl methacrylate, m-(p-methylphenylsulfonylamino)phenyl methacrylate, p-(p-methylphenylsulfonylamino)phenyl methacrylate, p-(α-naphthylsulfonylamino)phenyl methacrylate, p-(β-naphthylsulfonylamino)phenyl methacrylate, 2-methylsulfonylaminoethyl methacrylate, 2-ethylsulfonylaminoethyl methacrylate, 2-phenylsulfonylaminoethyl methacrylate, 2-p-methylphenylsulfonylaminoethyl methacrylate, 2-α-naphthylsulfonylaminoethyl methacrylate, 2-β-naphthylsulfonylaminoethyl methacrylate, o-dimethylaminosulfonylphenyl methacrylate, m-dimethylaminosulfonylphenyl methacrylate, p-dimethylaminosulfonylphenyl methacrylate, o-diethylaminosulfonylphenyl methacrylate, m-diethylaminosulfonylphenyl methacrylate, and p-diethylaminosulfonylphenyl methacrylate, and acrylic acid esters having the same substituents as above.

Examples further include p-aminosulfonylstyrene, p-aminosulfonyl-α-methylstyrene, p-aminosulfonylphenyl allyl ether, p-(N-methylaminosulfonyl)phenyl allyl ether, p-(N-dimethylaminosulfonyl)phenyl allyl ether, vinyl methylsulfonylaminoacetate, vinyl phenylsulfonylaminoacetate, allyl methylsulfonylaminoacetate, allyl phenylsulfonylaminoacetate, and p-methylsulfonylaminophenyl allyl ether.

Specific examples of Component A3 are shown below. The copolymerization ratio is expressed as mol %.

In the following chemical formulae Me denotes a methyl group, Ph denotes a phenyl group, Et denotes an ethyl group and Ac denotes an acetyl group.

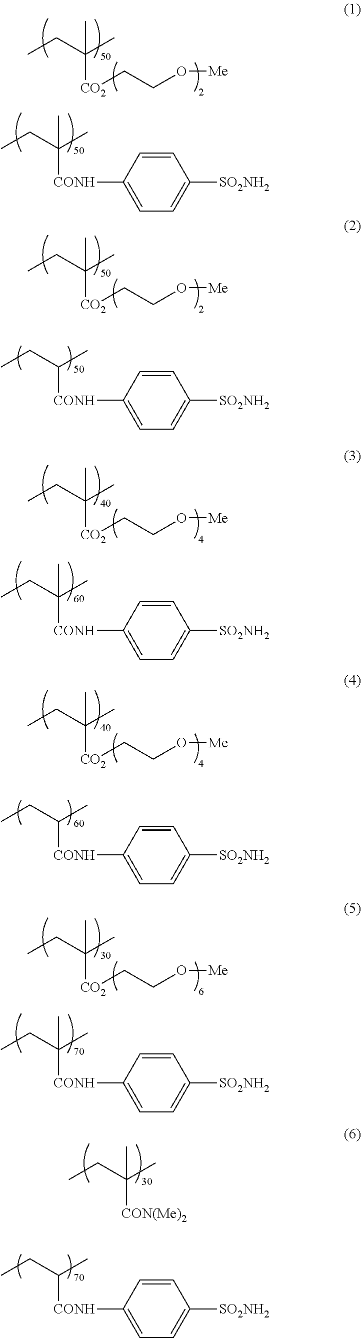

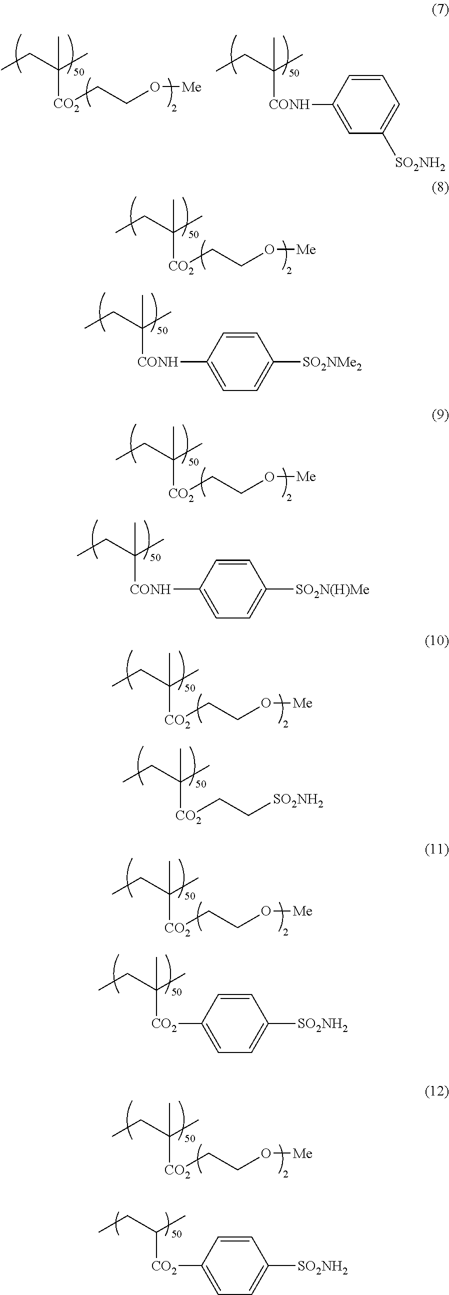

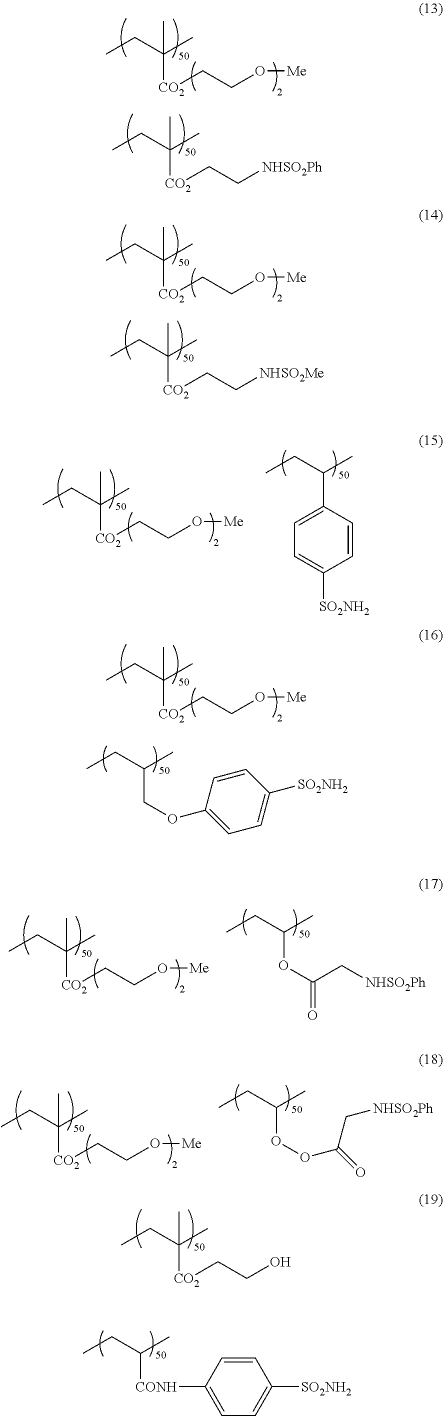

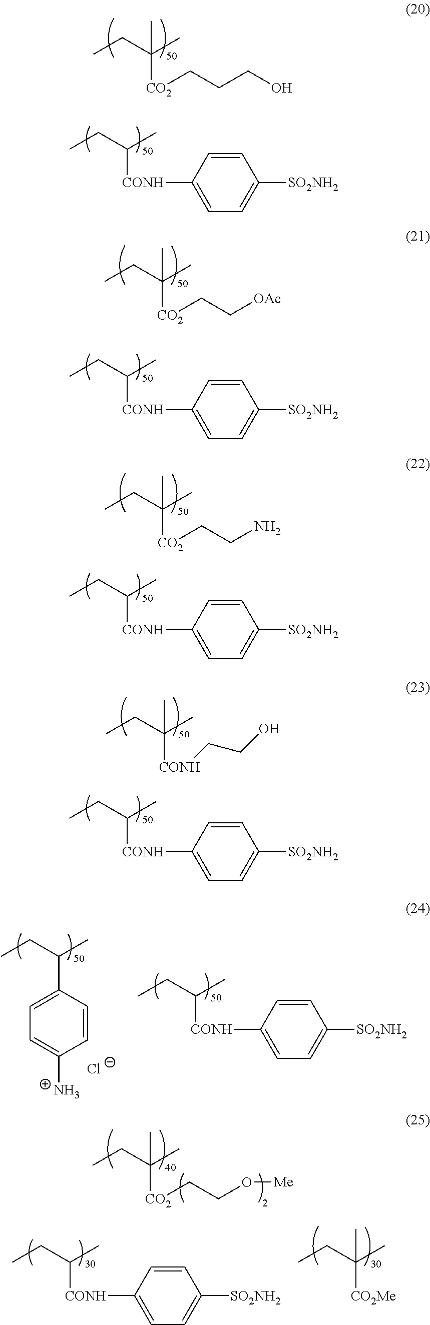

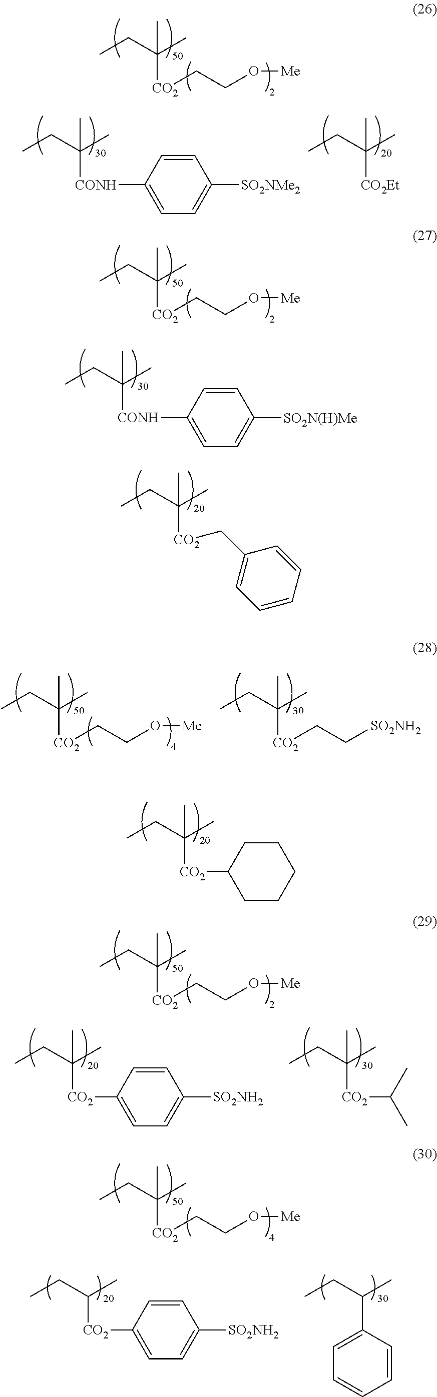

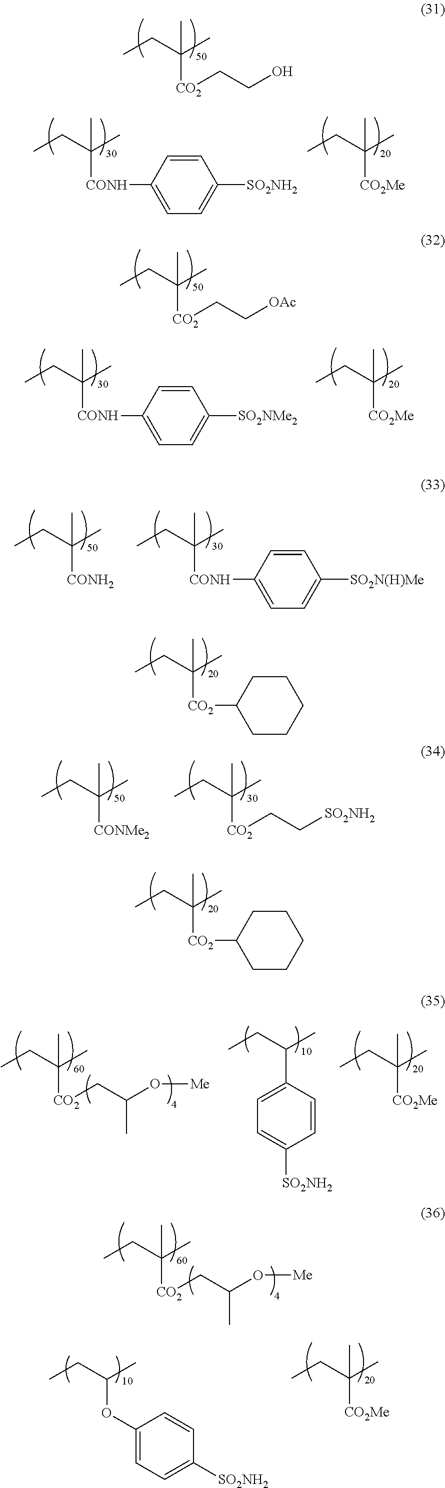

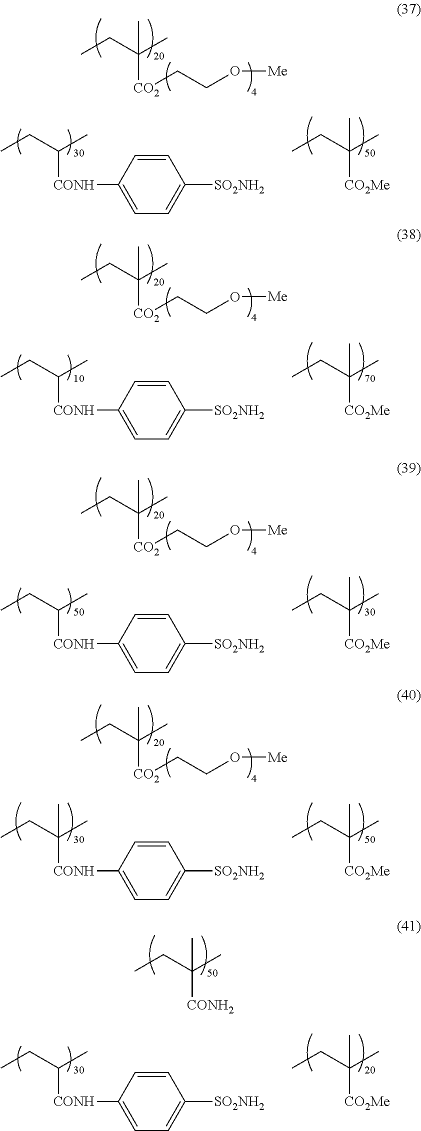

Examples of the low molecular weight compound containing a residue selected from the group consisting of a maleimide group, a succinimide group, and a phthalimide group (Component A2) are shown below.

N-(4-Aminophenyl)maleimide, N-[4-(2-benzimidazolyl)phenyl]maleimide, N-benzylmaleimide, N-biotinyl-N′-(3-maleimidopropionyl)-3,6-dioxaoctane-1,8-diamine, bis(3-ethyl-5-methyl-4-maleimidophenyl)methane, 4,4′-bismaleimidodiphenylmethane, 1,2-bis(maleimido)ethane,

1,6-bismaleimidohexane, 2,3-bis(2,4,5-trimethyl-3-thienyl)maleimide, N-bromomethyl-2,3-dichloromaleimide, N-cyclohexylmaleimide, 3,4-dibromomaleimide, N-ethylmaleimide, maleimide, 3-maleimidopropionic acid, 6,7-methylenedioxy-4-methyl-3-maleimidocoumarin, N-methylmaleimide, N-(4-nitrophenyl)maleimide, N,N′-1,3-phenylenedimaleimide, N,N′-1,4-phenylenedimaleimide, N-phenylmaleimide, N-(1-pyrenyl)maleimide, N-succinimidyl 3-maleimidobenzoate, N-succinimidyl 4-maleimidobutyrate, N-succinimidyl 6-maleimidohexanoate, N-succinimidyl 3-maleimidopropionate, N-(2,4,6-trichlorophenyl)maleimide, N-(allyloxycarbonyloxy)succinimide, N-bromosuccinimide, N-(tert-butoxycarbonyl)-O-benzyl-L-serine N-succinimidyl ester, N-(tert-butoxycarbonyl)-L-methionine N-succinimidyl ester, N-(tert-butoxycarbonyl)-D-proline succinimidyl ester, N-α-(tert-butoxycarbonyl)-L-tryptophan N-succinimidyl ester, N-carbobenzoxy-L-leucine N-succinimidyl ester, N-carbobenzoxy-L-valine succinimidyl ester, 2-bromobenzyl succinimidyl carbonate, N-chlorosuccinimide, di(N-succinimidyl carbonate, di(N-succinimidyl) 3,3′-dithiodipropionate, di(N-succinimidyl)suberate, N-[(9H-fluoren-9-ylmethoxy)carbonyloxy]succinimide, N-hydroxysuccinimide, N-hydroxysulfosuccinimide sodium salt, succinimide, N-succinimidyl acrylate, D-biotin N-succinimidyl ester, succinimidyl 6-[[7-(N,N-dimethylaminosulfonyl)-2,1,3-benzoxadiazol-4-yl]amino]hexanoate, succinimidyl 4-[3,5-dimethyl-4-(4-nitrobenzyloxy)phenyl]-4-oxobutyrate, N-succinimidyl 6-(2,4-dinitroanilino)hexanoate, N-succinimidyl 3-(diphenylphosphino)propionate, N-succinimidyl ferrocenecarboxylate, N-succinimidyl methacrylate, N-succinimidyl 4-nitrophenylacetate, N-succinimidyl 3-(2-pyridyldithio)propionate, N-succinimidyl S-acetylthioglycolate, succinimidyl (2R)-6-(tetrahydro-2H-pyran-2-yloxy)-2,5,7,8-tetramethylchroman-2-carboxylate, N-(1,2,2,2-tetrachloroethoxycarbonyloxy)succinimide, and N,N,N′,N′-tetramethyl-O—(N-succinimidyl)uronium tetrafluoroborate.

Other specific examples of the polymer compound containing a constituent unit derived from a maleimide group or a constituent unit containing a sulfonamide group are shown below. Me denotes a methyl group, n-Bu denotes an n-butyl group, and Ac denotes an acetyl group.

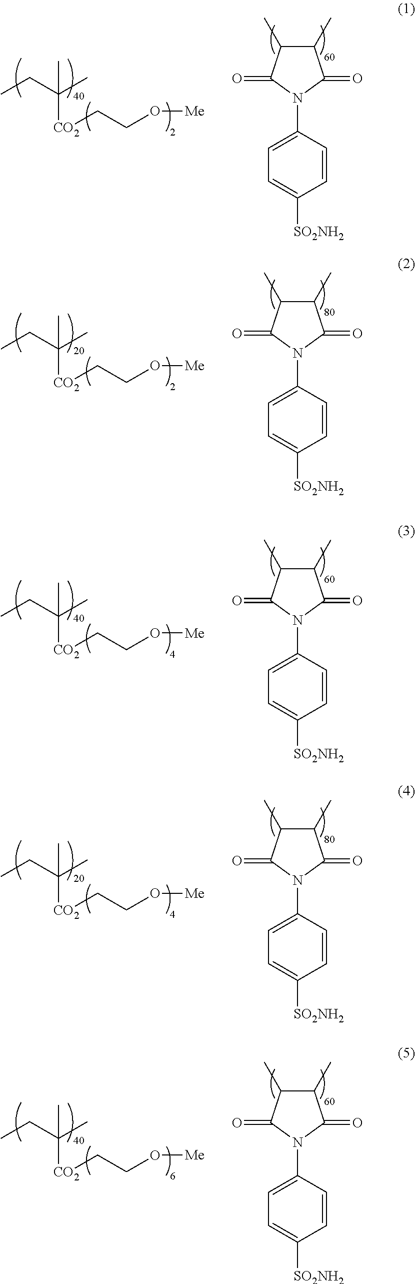

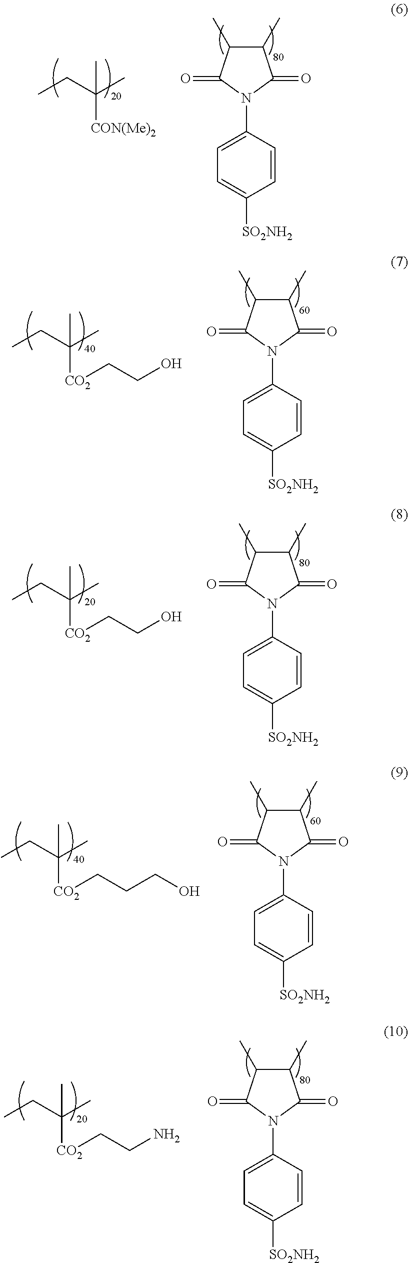

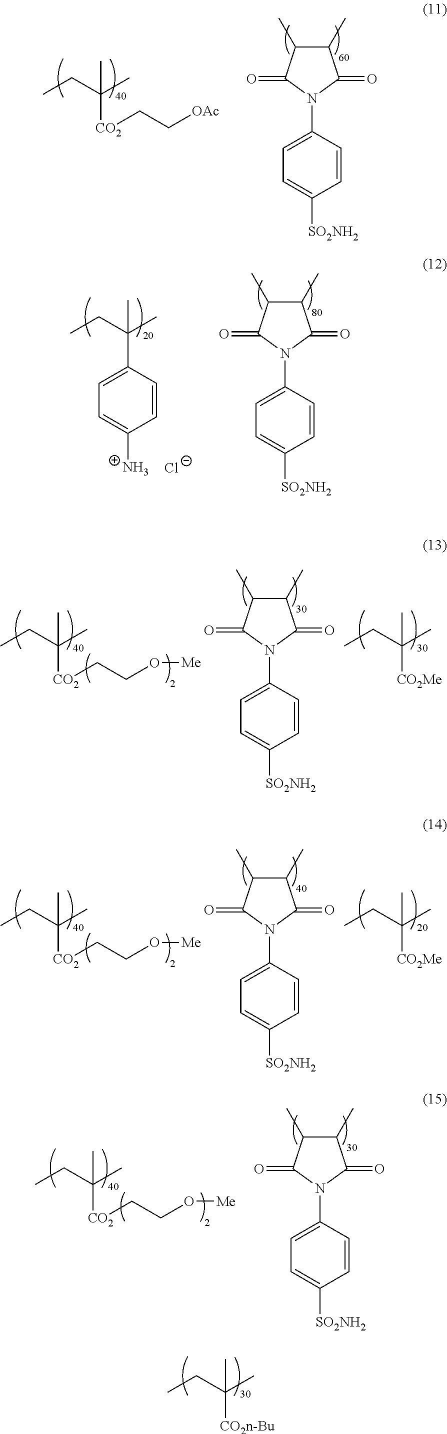

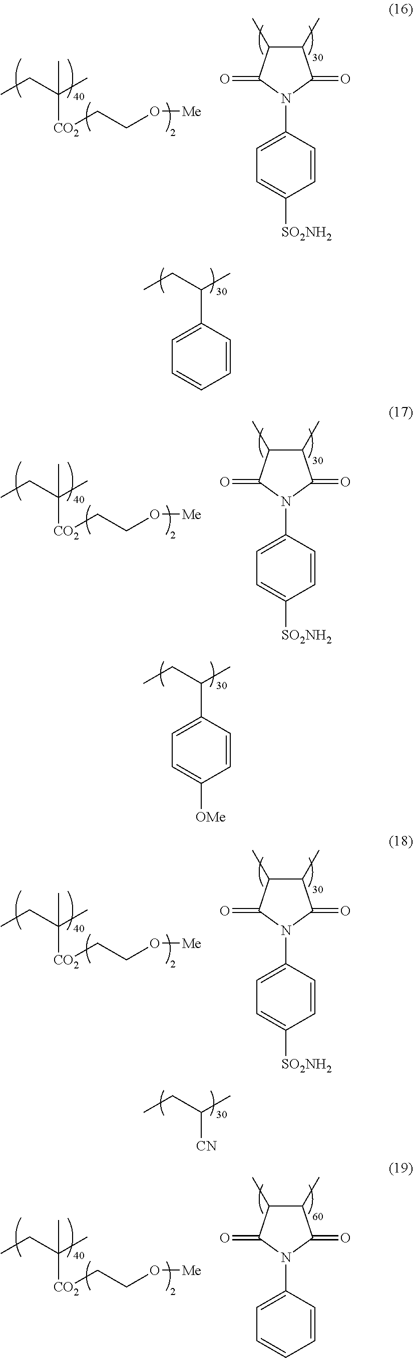

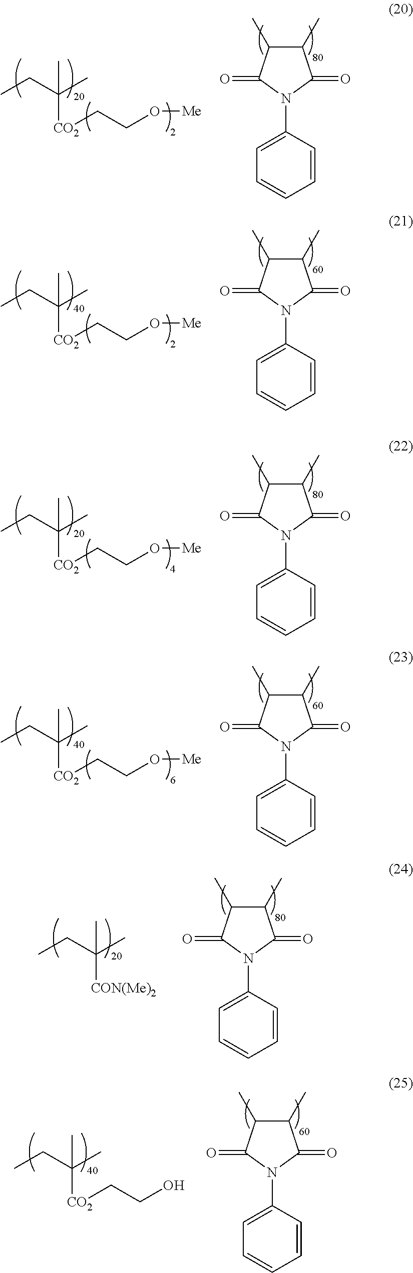

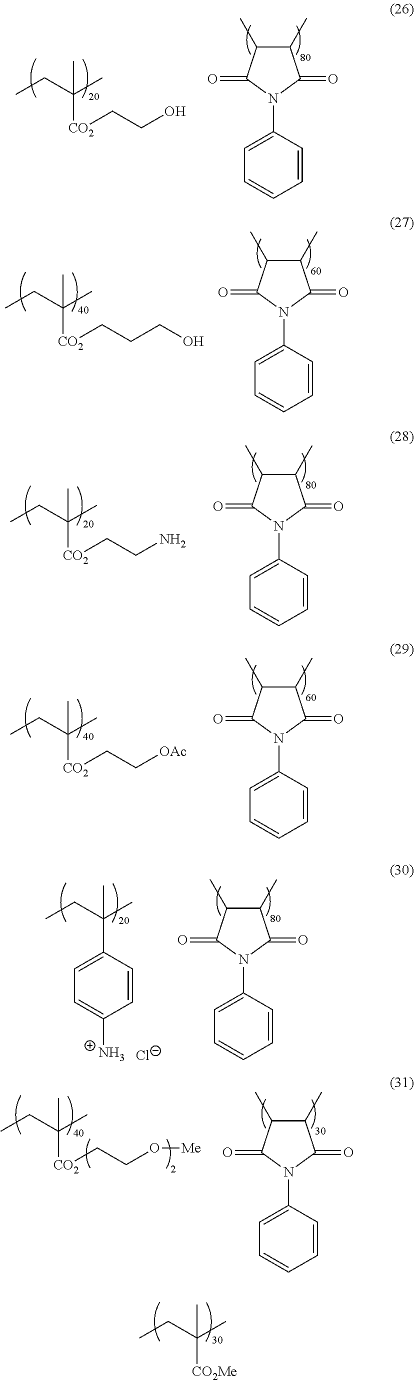

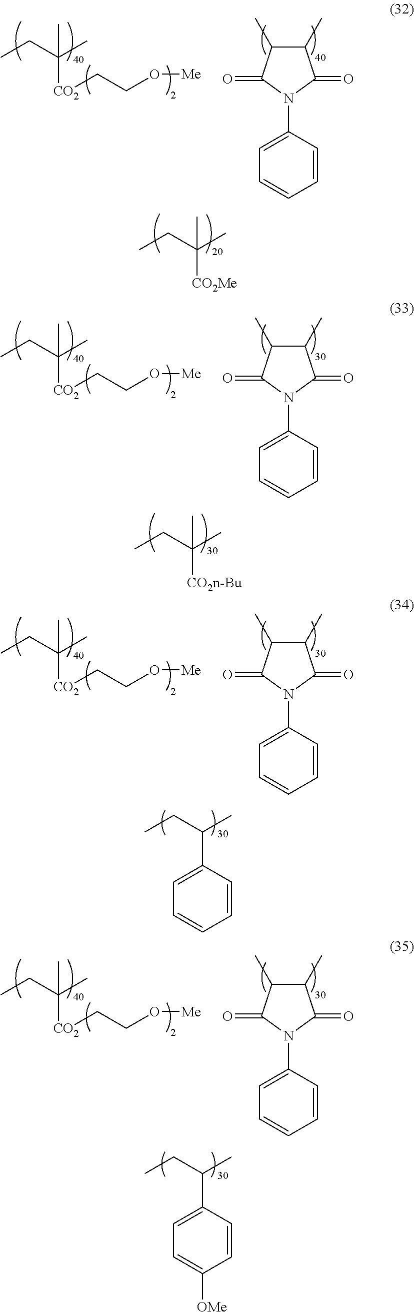

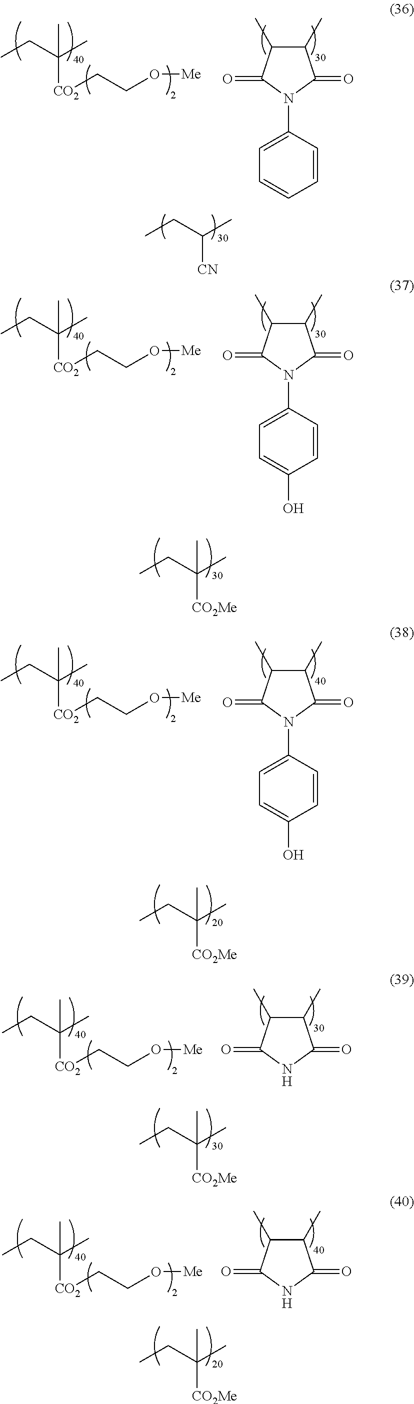

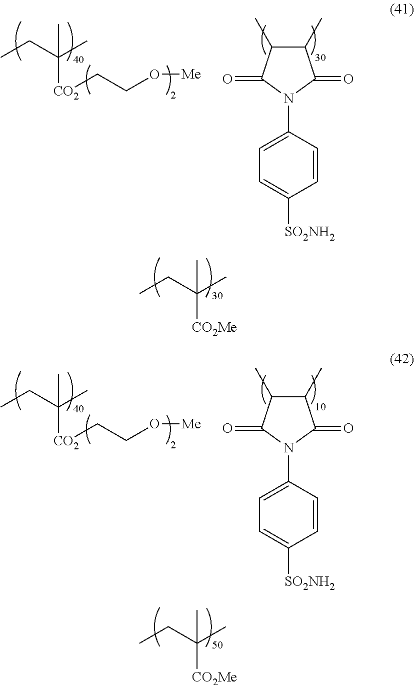

### <(Component B) Binder Polymer>

The resin composition of the present invention preferably comprises as an optional component (Component B) a binder polymer that is different from Component A above. That is, Component B is a polymer that is different from the polymer compound containing a constituent unit derived from a maleimide group or a constituent unit containing a sulfonamide group.

Furthermore, when Component A contains at least one type from a hydrolyzable silyl group and a silanol group, Component B preferably contains in the molecule a functional group that can react with the above group to form a crosslinked structure (hereinafter, called a ‘reactive functional group’ as appropriate), and such a binder polymer is called polymer (B) below as appropriate.

When Component A contains at least one type from a hydrolyzable silyl group and a silanol group, the reactive functional group that can react with Component A is not particularly limited as long as it can react with these groups to form an —Si—O— bond, and a hydroxy group, an alkoxy group, a silanol group, or a hydrolyzable silyl group is preferably used.

These functional groups may be present at any position of the polymer molecule of Component B, but are preferably present on a side chain of the polymer chain in particular. As such a polymer, a vinyl copolymer (copolymer of a vinyl monomer such as polyvinyl alcohol or polyvinyl acetal, or a derivative thereof) or an acrylic resin (copolymer of an acrylic monomer such as hydroxyethyl (meth)acrylate, or a derivative thereof) is preferably used. Here, the derivative of a copolymer of a vinyl monomer specifically means a binder polymer in which a side chain is extended by chemically modifying the OH group of the vinyl alcohol unit or the α-position relative to the OH group and introducing into the terminal a functional group that can react with Compound (A), such as an OH group or a carboxyl group. Furthermore, examples of the derivative of a copolymer of an acrylic monomer include a resin into which a functional group that can react with Compound (A), such as an OH group or a carboxyl group, is introduced.

A process for producing the binder polymer (Component B) that can be used in the present invention is not particularly limited, and examples thereof include a production process involving polymerization or copolymerization of a polymerizable monomer containing a group that can react with at least one of a hydrolyzable silyl group and a silanol group to form a crosslinked structure or a group that can be converted into the above group.

As such a polymer (B), in particular, (B-1) a binder polymer having a hydroxy group is preferably used.

### (Component B1) Binder Polymer Having a Hydroxyl Group

Hereinafter, (Component B1) a binder polymer having a hydroxyl group (hereinafter, appropriately also referred to as a “specific polymer (B-1)”) will be explained. This binder polymer is preferably insoluble in water and soluble in alcohol having 1 to 4 carbon atoms.

As specific polymer (B-1), from the view point of satisfying both good durability properties for an aqueous ink and for a UV ink, and having a high engraving sensitivity and good film performance, polyvinyl butyral (PVB) and derivatives thereof, acrylic resins having a hydroxyl group on a side chain, epoxy resins having a hydroxyl group on a side chain, etc. are preferable.

A specific polymer (B-1) used in the present invention is a preferable co-component for forming a laser-engraving resin composition in the present invention. Improvement of engraving sensitivity can be obtained when combined with a photothermal conversion agent (Component G) which can absorb light having a wavelength of 700 to 1,300 nm described below and making a glass transition temperature (Tg) of at least 20° C. A binder polymer having such a glass transition temperature is also called a non-elastomer below. That is, generally, an elastomer is academically defined as a polymer having a glass transition temperature of no greater than 20° C. (room temperature) (ref. Kagaku Dai Jiten 2nd edition (Science Dictionary), Foundation for Advancement of International Science, Maruzen, page 154). Non-elastomer refers to a polymer which a glass transition temperature of greater than room temperature. The upper limit for the glass transition temperature of the polymer is not limited, but is preferably no greater than 200° C. from the viewpoint of ease of handling, and is more preferably at least 25° C. but no greater than 120° C.

When a polymer having a glass transition temperature of room temperature (20° C.) or greater is used, a specific polymer (B-1) is in a glass state at normal temperature. Because of this, compared with a case of the rubber state, thermal molecular motion is suppressed. In laser engraving, in addition to the heat given by a laser during laser irradiation, heat generated by the function of (Component G) a photothermal conversion agent added as desired is transmitted to the surrounding specific polymer (B-1), and this polymer is thermally decomposed and disappears, thereby forming an engraved recess.

In preferred mode of the present invention, it is surmised that when (Component G) a photothermal conversion agent is present in a state in which thermal molecular motion of a specific polymer (B-1) is suppressed, heat transfer to and thermal decomposition of the specific polymer (B-1) occur effectively. It is anticipated that such an effect further increases the engraving sensitivity.

Specific examples of specific polymers (B-1) that are non-elastomer for use preferably in the present invention are cited below.

### (1) Polyvinyl Acetal and its Derivative

In this description, hereinafter, polyvinyl acetal and derivatives thereof are called just a polyvinyl acetal derivative. That is, a polyvinyl acetal derivative includes polyvinyl acetal and derivatives thereof, and is a generic term used to refer to compounds obtained by converting polyvinyl alcohol (obtained by saponifying polyvinyl acetate) into a cyclic acetal.

The acetal content in the polyvinyl acetal derivative (mol % of vinyl alcohol units converted into acetal relative to the total number of moles of vinyl acetate monomer starting material as 100 mol %) is preferably 30 to 90 mol %, more preferably 50 to 85 mol %, and particularly preferably 55 to 78 mol %.

The vinyl alcohol unit in the polyvinyl acetal is preferably 10 to 70 mol % relative to the total number of moles of the vinyl acetate monomer starting material, more preferably 15 to 50 mol %, and particularly preferably 22 to 45 mol %.

Furthermore, the polyvinyl acetal may have a vinyl acetate unit as another component, and the content thereof is preferably 0.01 to 20 mol %, and more preferably 0.1 to 10 mol %. The polyvinyl acetal derivative may further have another copolymerized constitutional unit.

Examples of the polyvinyl acetal derivative include a polyvinyl butyral derivative, a polyvinyl propylal derivative, a polyvinyl ethylal derivative, and a polyvinyl methylal derivative. Among them, a polyvinyl butyral derivative (hereinafter, it is also referred to as a “PVB derivative”) is a derivative that is preferable. In this description, for examples, a polyvinyl butyral derivative includes polyvinyl butyral and derivatives thereof, and the same can be said for other polyvinyl acetal derivatives.

From the viewpoint of a balance being achieved between engraving sensitivity and film formation properties, the weight-average molecular weight of the polyvinyl acetal derivative is preferably 5,000 to 800,000, more preferably 8,000 to 500,000 and, from the viewpoint of improvement of rinsing properties for engraving residue, particularly preferably 50,000 to 300,000.

Preferable examples of a polyvinyl butyral derivative are cited for explanation, but there are not limited to these.

An example of structure of polyvinyl butyral derivatives is shown below, and is constituted while comprising these constitutional units.

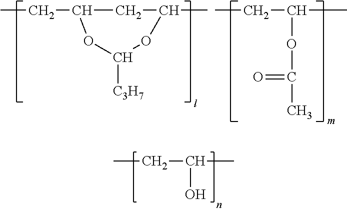

In the Formula above, l, m, and n denote the contents (mol %) of the respective repeating units contained in polyvinylbutyral of the Formula above, the relationship l+m+n=100 being satisfied. The butyral content in the polyvinylbutyral or derivative thereof (value for l in the Formula above) is preferably 30 to 90 mol %, more preferably 40 to 85 mol %, and particularly preferably 45 to 78 mol %.

From the viewpoint of a balance being achieved between engraving sensitivity and film properties, the weight-average molecular weight of the polyvinylbutyral or derivative thereof is preferably 5,000 to 800,000, and more preferably 8,000 to 500,000, and from the viewpoint of improving rinsing properties for engraving residue it is particularly preferably 50,000 to 300,000.

Derivatives of PVB are available as a commercial product. As specific examples, from the viewpoint of alcohol (in particular, ethanol) solubility, “Eslec B” series and “Eslec K (KS)” series (Sekisui Chemical Co., Ltd.) and “Denka Butyral” (Denki Kagaku Kogyo Kabushiki Kaisha) are preferable, and, from the viewpoint of alcohol (in particular, ethanol) solubility, “Eslec B” series (Sekisui Chemical Co., Ltd.) and “Denka Butyral” (Denki Kagaku Kogyo Kabushiki Kaisha) are more preferable.

Among these, particularly preferable commercial products are shown below with values of l, m and n in Formula above and molecular weight. With regard to “Eslec B” series (Sekisui Chemical Co., Ltd.), “BL-1” (l=61, m=3, n=36, weight average molecular weight: 19,000), “BL-1H” (l=67, m=3, n=30, weight average molecular weight: 20,000), “BL-2” (l=61, m=3, n=36, weight average molecular weight: about 27,000), “BL-5” (l=75, m=4, n=21, weight average molecular weight: 32,000), “BL-S” (l=74, m=4, n=22, weight average molecular weight: 23,000), “BM-S” (l=73, m=5, n=22, weight average molecular weight: 53,000), “BH-S” (l=73, m=5, n=22, weight average molecular weight: 66,000) are cited. With regard to “Denka Butyral” series (Denki Kagaku Kougyo Kabushiki Kaisha), “#3000-1” (l=71, m=1, n=28, weight average molecular weight: 74,000), “#3000-2” (l=71, m=1, n=28, weight average molecular weight: 90,000), “#3000-4” (l=71, m=1, n=28, weight average molecular weight: 117,000), “#4000-2” (l=71, m=1, n=28, weight average molecular weight: 152,000), “#6000-C” (l=64, m=1, n=35, weight average molecular weight: 308,000), “#6000-EP” (l=56, m=15, n=29, weight average molecular weight: 381,000), “#6000-CS” (l=74, m=1, n=25, weight average molecular weight: 322,000), “#6000-AS” (l=73, m=1, n=26, weight average molecular weight: 242,000) are cited.

When the relief-forming layer is formed using PVB as the specific polymer (B-1), a method of casting and drying a solution prepared by solving it in a solvent is preferable from the viewpoint of the flatness of the film surface.

### (2) An Acrylic Resin

As acrylic resin for use as specific polymer (B-1) of the present invention, an acrylic resin may be used which can be obtainable from known acrylic monomers having a hydroxyl group in the molecule.

Preferable examples of the acrylic monomer having a hydroxyl group include a (meth)acrylic acid ester, a crotonic acid ester, or a (meth)acrylamide that has a hydroxyl group in the molecule. Specific examples of such a monomer include 2-hydroxyethyl (meth)acrylate, 2-hydroxypropyl (meth)acrylate, and 4-hydroxybutyl (meth)acrylate.

As acrylic resin, the acrylic monomer other than that having hydroxy group may comprises as a co-monomer. Examples thereof such an acrylic monomer include, as the (meth)acrylic ester, methyl (meth)acrylate, ethyl (meth)acrylate, n-propyl(meth)acrylate, isopropyl(meth)acrylate, n-butyl (meth)acrylate, isobutyl (meth)acrylate, tert-butyl (meth)acrylate, n-hexyl (meth)acrylate, 2-ethylhexyl(meth)acrylate, acetoxyethyl (meth)acrylate, phenyl(meth)acrylate, 2-methoxyethyl (meth)acrylate, 2-ethoxyethyl (meth)acrylate, 2-(2-methoxyethoxy)ethyl (meth)acrylate, cyclohexyl (meth)acrylate, benzyl (meth)acrylate, diethylene glycol monomethyl ether (meth)acrylate, diethylene glycol monoethyl ether (meth)acrylate, diethylene glycol monophenyl ether (meth)acrylate, triethylene glycol monomethyl ether (meth)acrylate, triethylene glycol monoethyl ether (meth)acrylate, dipropylene glycol monomethyl ether (meth)acrylate, polyethylene glycol monomethyl ether (meth)acrylate, polypropylene glycol monomethyl ether (meth)acrylate, the monomethyl ether (meth)acrylate of a copolymer of ethylene glycol and propylene glycol, N,N-dimethylaminoethyl (meth)acrylate, N,N-diethylaminoethyl (meth)acrylate, and N,N-dimethylaminopropyl (meth)acrylate.

Furthermore, a modified acrylic resin formed with a urethane group- or urea group-containing acrylic monomer may preferably be used.

Among these, from the viewpoint of aqueous ink resistance, an alkyl (meth)acrylate such as lauryl(meth)acrylate and an aliphatic cyclic structure-containing (meth)acrylate such as t-butylcyclohexyl(meth)acrylate are particularly preferable.

Furthermore, as the specific polymer (B-1), a novolac resin may be used, this being a resin formed by condensation of a phenol and an aldehyde under acidic conditions.

Preferred examples of the novolac resin include a novolac resin obtained from phenol and formaldehyde, a novolac resin obtained from m-cresol and formaldehyde, a novolac resin obtained from p-cresol and formaldehyde, a novolac resin obtained from o-cresol and formaldehyde, a novolac resin obtained from octylphenol and formaldehyde, a novolac resin obtained from mixed m-/p-cresol and formaldehyde, and a novolac resin between a mixture of phenol/cresol (any of m-, p-, o- or m-/p-, m-/o-, o-/p-mixtures) and formaldehyde.

With regard to these novolac resins, those having a weight-average molecular weight of 800 to 200,000 and a number-average molecular weight of 400 to 60,000 are preferable.

An epoxy resin having a hydroxy group in a side chain may be used as the specific polymer (B-1). A preferred example of the epoxy resin is an epoxy resin formed by polymerization, as a starting material monomer, of an adduct of bisphenol A and epichlorohydrin. The epoxy resin preferably has a weight-average molecular weight of at least 800 but no greater than 200,000, and a number-average molecular weight of at least 400 but no greater than 60,000.

Among specific polymers (B-1), polyvinyl butyral derivatives are particularly preferable from the viewpoint of rinsing properties and printing durability when the polymer is formed into the relief-forming layer.

In (Component B) polymers of any embodiment described above, the content of the hydroxyl group contained in the specific polymer in the present invention is preferably 0.1 to 15 mmol/g, and more preferably 0.5 to 7 mmol/g.

Component B-1 in the resin composition of the present invention may be used only in one kind, or in two or more kinds in combination.

### (Component B-2) Binder Polymer for Use in Combination

The resin composition for laser engraving of the present invention may comprise as (Component B) a binder polymer (B-2) a binder polymer for use in combination, which is known and not included as Component B1. Such binder polymers are referred as (B-2) binder polymer for use in combination.

The binder polymer (B-2) is a primary component of the resin composition for laser engraving in addition to specific polymer (B-1) above, and a general polymer compound not classified as specific polymer (B-1) may be selected appropriately and used singly or in combination of two or more types. In particular, when the resin composition for laser engraving is to be used as a relief printing plate precursor for laser engraving, preferably the selection is performed while considering various performances such as laser engraving properties, ink-adhering properties, and dispersion properties of engraving residue.

The binder polymer for use in combination (B-2) may be selected and used from polystyrene resin, polyester resin, polyamide resin, polysulfone resin, polyethersulfone resin, polyimide resin, hydrophilic polymer comprising a hydroxyethylene unit, acrylic resin, acetal resin, epoxy resin, polycarbonate resin, rubber, thermoplastic elastomer, etc.

For example, from the viewpoint of laser engraving sensitivity, a polymer comprising a partial structure that is thermally decomposed by exposure or heating is preferable. As such polymer, those described in JP-A-2008-163081, paragraph 0038 are preferably cited. Moreover, when a purpose is to form a film that has softness and flexibility, a soft resin or a thermoplastic elastomer is selected. There is detailed description in JP-A-2008-163081, paragraphs 0039 to 0040. Furthermore, in the case where the resin composition for laser engraving is applied to the relief-forming layer in the relief printing plate precursor for laser engraving, from the viewpoint of easiness of preparing a composition for the relief-forming layer and improvement of resistance properties for an oil-based ink in the relief printing plate to be obtained, the use of a hydrophilic or alcoholphilic polymer is preferable. As the hydrophilic polymer, those described in detail in JP-A-2008-163081, paragraph 0041 can be used.

Furthermore, a polyester containing a hydroxycarboxylic acid unit such as polylactic acid may preferably be used. Specifically, such a polyester is preferably selected from the group consisting of a polyhydroxyalkanoate (PHA), a lactic acid-based polymer, polyglycolic acid (PGA), polycaprolactone (PCL), poly(butylenesuccinic acid), and derivatives and mixtures thereof.

Similarly, when it is used for the purpose of curing by heat or light exposure and improving strength, a polymer having a carbon-carbon unsaturated bond in the molecule is preferably used.

As a polymer having a carbon-carbon unsaturated bond in the main chain, SI (polystyrene-polyisoprene), SB (polystyrene-polybutadiene), SBS (polystyrene-polybutadiene-polystyrene), SIS (polystyrene-polyisoprene-polystyrene), SEBS (polystyrene-polyethylene/polybutylene-polystyrene), etc. can be cited.

A polymer having a carbon-carbon unsaturated bond in a side chain may be obtained by introducing, into a side chain of the skeleton of the binder polymer applicable in the present invention, a carbon-carbon unsaturated bond such as an allyl group, an acryloyl group, a methacryloyl group, a styryl group, or a vinyl ether group. As a method for introducing a carbon-carbon unsaturated bond into a binder polymer side chain, a known method such as a method in which a polymer is copolymerized with a structural unit having a polymerizable group precursor formed by bonding a protecting group to a polymerizable group, and the protecting group is removed to give a polymerizable group or a method in which a polymer compound having a plurality of reactive groups such as hydroxy groups, amino groups, epoxy groups, or carboxy groups is prepared and a polymer reaction is carried out with a compound having a carbon-carbon unsaturated bond and a group that reacts with these reactive groups may be employed. In accordance with these methods, the amount of unsaturated bond and polymerizable group introduced into the polymer compound can be controlled.

As described above, the binder polymer may be used singly or in combination of two or more taking into consideration physical properties in accordance with the use application of relief printing plate and selecting them for the purpose.

The weight-average molecular weight (polystyrene basis by GPC measurement) of the binder polymer in the present invention is preferably 5,000 to 500,000. When the weight-average molecular weight is at least 5,000, the shape retention as a single resin is excellent, and when it is no greater than 500,000, it is easily dissolved in a solvent such as water and it is convenient for preparation of the relief-forming layer. The weight-average molecular weight of the binder polymer is more preferably 10,000 to 400,000, and particularly preferably 15,000 to 300,000.

The total content of the binder polymer (sum total of contents of Component B) is preferably 5 to 95 wt % relative to a solids content basis total weight of the resin composition for laser engraving, more preferably 15 to 80 wt %, and yet more preferably 20 to 65 wt %.

For example, when the resin composition for laser engraving of the present invention is applied to the relief-forming layer of the relief printing plate precursor, setting the content of the binder polymer to at least 5 wt % gives printing durability that is sufficient for the relief printing plate so obtained to be used as a printing plate, and setting it to no greater than 80 wt % gives flexibility that is sufficient for the relief printing plate so obtained to be used as a flexographic printing plate, without making other components insufficient.

The relief-forming layer related to the present invention is preferably formed from a resin composition (the resin composition of the present invention) comprising Component A, which is described above as an essential component in the resin composition of the present invention, Component B (polymer binder), which is used as desired, and an optional component such as a polymerizable compound, a polymerization initiator, or a plasticizer. and beyond are described in detail below.

### (Component C) Polymerizable Compound

In the present invention, from the viewpoint of forming a crosslinked structure in the relief-forming layer, the coating solution for the relief-forming layer (the resin composition of the present invention) preferably comprises a polymerizable compound.

Polymerizable compounds that can be used here can be broadly divided into (Component C1) a monofunctional monomer having one ethylenically unsaturated group in the molecule and a polyfunctional monomer having two or more of the above group in the molecule, and (Component C2) a compound having at least one type from a hydrolyzable silyl group and a silanol group. Compounds having a plurality of these crosslinkable groups have crosslinkablility and are effective in enhancing the resistance of the relief layer toward the ink.

The polymerizable compound (Component C) is a compound other than Component A, and preferably has no sulfonamide group and also no maleimide group.

### (Component C1) Monofunctional Monomer Containing One Ethylenically Unsaturated Group in Molecule and Polyfunctional Monomer Containing Two or More of Above Group in Molecule

Component C1 above used as a polymerizable compound is now explained.

Since the relief-forming layer related to the present invention preferably has a crosslinked structure in the film, a polyfunctional monomer is preferably used compared with a monofunctional monomer. The molecular weight of such a polyfunctional monomer is preferably 200 to 2,000. The ethylenically unsaturated group is preferably an acyclic group.

Examples of the monofunctional monomer include an ester of an unsaturated carboxylic acid (e.g. acrylic acid, methacrylic acid, itaconic acid, crotonic acid, isocrotonic acid, maleic acid, etc.) and a monohydric alcohol compound, and an amide of an unsaturated carboxylic acid and a monoamine compound. Examples of the polyfunctional monomer include an ester of an unsaturated carboxylic acid (e.g. acrylic acid, methacrylic acid, itaconic acid, crotonic acid, isocrotonic acid, maleic acid, etc.) and a polyhydric alcohol compound, and an amide of an unsaturated carboxylic acid and a polyamine compound.

Furthermore, an addition reaction product of an unsaturated carboxylic acid ester or amide having a nucleophilic substituent such as a hydroxy group, an amino group, or a mercapto group with a monofunctional or polyfunctional isocyanate or epoxy, a dehydration-condensation reaction product with a monofunctional or polyfunctional carboxylic acid, etc. are also suitably used.

Furthermore, as the polymerizable compound having an ethylenically unsaturated group, addition products of unsaturated carboxylic acid esters or amides having an electrophilic substituent such as an isocyanate group or an epoxy group with a monofunctional or polyfunctional alcohol, amine or thiol, and substitution products of unsaturated carboxylic acid esters or amides having a leaving substituent such as a halogeno group or a tosyloxy group with a monofunctional or polyfunctional alcohol, amine or thiol are also preferable.

In addition, as other examples, the use of compounds obtained by substituting the unsaturated carboxylic acid by an unsaturated sulfonic acid, styrene, a vinyl ether or the like is also possible.

The polymerizable compound having an ethylenically unsaturated group is not particularly limited, and, in addition to compounds exemplified above, various known compounds may be used. For example, compounds described in JP-A-2009-204962, paragraphs 0098 to 0124 may be used.

From the viewpoint of improving engraving sensitivity, it is preferable in the present invention to use as the polymerizable compound a compound having a sulfur atom in the molecule.

As such an ethylenically unsaturated compound having a sulfur atom in the molecule, it is preferable from the viewpoint of improving engraving sensitivity in particular to use a polymerizable compound having two or more ethylenically unsaturated bonds and having a carbon-sulfur bond at a site where two ethylenically unsaturated bonds among them are linked (hereinafter, referred as a ‘sulfur-containing polyfunctional monomer’ as appropriate).

Examples of carbon-sulfur bond-containing functional groups of the sulfur-containing polyfunctional monomer in the present invention include sulfide, disulfide, sulfoxide, sulfonyl, sulfonamide, thiocarbonyl, thiocarboxylic acid, dithiocarboxylic acid, sulfamic acid, thioamide, thiocarbamate, dithiocarbamate, and thiourea-containing functional groups.

Furthermore, a linking group containing a carbon-sulfur bond linking two ethylenically unsaturated bonds of the sulfur-containing polyfunctional monomer is preferably at least one unit selected from —C—S—, —C—S—S—, —NHC(═S)O—, —NHC(═O)S—, —NHC(═S)S—, and —C—SO₂—.

Moreover, the number of sulfur atoms contained in the sulfur-containing polyfunctional monomer molecule is not particularly limited as long as it is one or more, and may be selected as appropriate according to the intended application, but from the viewpoint of a balance between engraving sensitivity and solubility in a coating solvent it is preferably 1 to 10, more preferably 1 to 5, and yet more preferably 1 or 2.

On the other hand, the number of ethylenically unsaturated bond sites contained in the molecule is not particularly limited as long as it is two or more and may be selected as appropriate according to the intended application, but from the viewpoint of flexibility of a crosslinked film it is preferably 2 to 10, more preferably 2 to 6, and yet more preferably 2 to 4.

From the viewpoint of flexibility of a film that is formed, the molecular weight of the sulfur-containing polyfunctional monomer in the present invention is preferably 120 to 3,000, and more preferably 120 to 1,500.

Furthermore, the sulfur-containing polyfunctional monomer in the present invention may be used on its own or as a mixture with a polyfunctional polymerizable compound or monofunctional polymerizable compound having no sulfur atom in the molecule.

Moreover, examples of the polymerizable compound having a sulfur atom in the molecule include those described in paragraphs 0032 to 0037 of JP-A-2009-255510.

From the viewpoint of engraving sensitivity, a mode in which a sulfur-containing polyfunctional monomer is used on its own or a mixture of a sulfur-containing polyfunctional monomer and a monofunctional ethylenic monomer is used is preferable, and a mode in which a mixture of a sulfur-containing polyfunctional monomer and a monofunctional ethylenic monomer is used is more preferable.

Another polymerizable compound that can be used as Component C may be selected from (Component C2) a compound containing at least one type from a hydrolyzable silyl group and a silanol group, and may be selected from compounds having at least one trialkoxysilyl group, preferably two or more, and more preferably 2 to 6. Such a polymerizable compound is explained below.

### <(Component C2) Compound Having at Least One of a Hydrolyzable Silyl Group and a Silanol Group>

The ‘hydrolyzable silyl group’ of (Component C2) a compound having at least one of a hydrolyzable silyl group and a silanol group (hereinafter, called as appropriately ‘(Component C2)’) used in the resin composition for laser engraving of the present invention is a silyl group that is hydrolyzable; examples of hydrolyzable groups include an alkoxy group, a mercapto group, a halogen atom, an amide group, an acetoxy group, an amino group, and an isopropenoxy group. A silyl group is hydrolyzed to become a silanol group, and a silanol group undergoes dehydration-condensation to form a siloxane bond. Such a hydrolyzable silyl group or silanol group is preferably one represented by Formula (1) below.

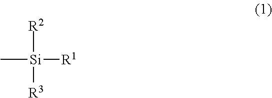

In Formula (1) above, R¹ to R³ denote independently a hydrolyzable group selected from the group consisting of an alkoxy group, an aryloxy group, a mercapto group, a halogen atom, an amide group, an acetoxy group, an amino group, and an isopropenoxy group, or a hydroxy group, a hydrogen atom, or a monovalent organic group. At least one of R¹ to R³ denotes a hydrolyzable group selected from the group consisting of an alkoxy group, an aryloxy group, a mercapto group, a halogen atom, an amide group, an acetoxy group, an amino group, and an isopropenoxy group, or a hydroxy group.

When R¹ to R³ denote a monovalent organic group, from the viewpoint that solubility in various types of organic solvents can be given, an organic group is preferably an alkyl group having 1 to 30 carbon atoms.

In Formula (1) above, the hydrolyzable group bonded to the silicon atom is particularly preferably an alkoxy group or a halogen atom.

From the viewpoint of rinsing properties and printing durability, the alkoxy group is preferably an alkoxy group having 1 to 30 carbon atoms, more preferably an alkoxy group having 1 to 15 carbon atoms, yet more preferably an alkoxy group having 1 to 5 carbon atoms, and particularly preferably an alkoxy group having 1 to 3 carbon atoms.

Furthermore, examples of the halogen atom include a F atom, a Cl atom, a Br atom, and a I atom, and from the viewpoint of ease of synthesis and stability it is preferably a Cl atom or a Br atom, and more preferably a Cl atom.

‘(Component C2) Compound having at least one of a hydrolyzable silyl group and a silanol group’ in the present invention is preferably a compound having one or more groups represented by Formula (1) above, and more preferably a compound having two or more. A compound having two or more hydrolyzable silyl groups is particularly preferably used. That is, a compound having in the molecule two or more silicon atoms having a hydrolyzable group bonded thereto is preferably used. The number of silicon atoms having a hydrolyzable group bond thereto contained in the compound is preferably at least 2 but no greater than 6, and most preferably 2 or 3.

A range of 1 to 3 of the hydrolyzable groups above may bond to one silicon atom, and the total number of hydrolyzable groups in Formula (1) is preferably in a range of 2 or 3. It is particularly preferable that three hydrolyzable groups are bonded to a silicon atom. When two or more hydrolyzable groups are bonded to a silicon atom, they may be identical to or different from each other.

Examples of the alkoxy group above include a methoxy group, an ethoxy group, a propoxy group, an isopropoxy group, a butoxy group, a tert-butoxy group, and a benzyloxy group. Examples of the alkoxysilyl group having an alkoxy group bonded thereto include a trialkoxysilyl group such as a trimethoxysilyl group, a triethoxysilyl group, a triisopropoxysilyl group, or a triphenoxysilyl group; a dialkoxymonoalkylsilyl group such as a dimethoxymethylsilyl group or a diethoxymethylsilyl group; and a monoalkoxydialkylsilyl group such as a methoxydimethylsilyl group or an ethoxydimethylsilyl group. A plurality of each of these alkoxy groups may be used in combination, or a plurality of different alkoxy groups may be used in combination.

Examples of the aryloxy group above include phenoxy group. Examples of the aryloxysilyl group having an aryloxy group bonded thereto include a triarylsilyl group such as a triphenylsilyl group.

Specific preferred examples of Component C2 compounds in the present invention include a compound in which a plurality of groups represented by Formula (1) above are bonded via a divalent linking group, and from the viewpoint of the effect, such a divalent linking group is preferably a linking group having a sulfide group, an imino group or a ureylene group.

A representative synthetic method of (Component C2) a compound containing a special linking group above is shown below.

<Synthetic Method for Compound Having Sulfide Group as Linking Group and Having Hydrolyzable Silyl Group and/or Silanol Group>

A synthetic method for the compound (C2) having a sulfide group as a linking group (hereinafter, called as appropriate a ‘sulfide linking group-containing C2 compound’) is not particularly limited, but can be synthesized by one of a synthetic method selected from the group comprising reaction of compound (C2) having a halogenated hydrocarbon group with an alkali metal sulfide, reaction of compound (C2) having a mercapto group with a halogenated hydrocarbon, reaction of compound (C2) having a mercapto group with compound (C2) having a halogenated hydrocarbon group, reaction of compound (C2) having a halogenated hydrocarbon group with a mercaptan, reaction of compound (C2) having an ethylenically unsaturated double bond with a mercaptan, reaction of compound (C2) having an ethylenically unsaturated double group with compound (C2) having a mercapto group, reaction of a compound having an ethylenically unsaturated group with compound (C2) having a mercapto group, reaction of a ketone with compound (C2) having a mercapto group, reaction of a diazonium salt with compound (C2) having a mercapto group, reaction of compound C2 having a mercapto group with an oxirane, reaction of compound (C2) having a mercapto group with compound (C2) having an oxirane group, reaction of a mercaptan with compound (C2) having an oxirane group, and reaction of compound (C2) having a mercapto group with an aziridine.

<Synthetic Method for Compound Having Imino Group as Linking Group and Having Hydrolyzable Silyl Group and/or Silanol Group>

A synthetic method for the compound (C2) having an imino group as a linking group (hereinafter, called as appropriate an ‘imino linking group-containing C2 compound’) is not particularly limited, but can be synthesized by one of a synthetic method selected from the group comprising reaction of compound (C2) having an amino group with a halogenated hydrocarbon, reaction of compound (C2) having an amino group with compound (C2) having a halogenated hydrocarbon group, reaction of compound (C2) having a halogenated hydrocarbon group with an amine, reaction of compound (C2) having an amino group with an oxirane, reaction of Component C having an amino group with compound (C2) having an oxirane group, reaction of an amine with the compound (C2) having an oxirane group, reaction of compound (C2) having an amino group with an aziridine, reaction of compound (C2) having an ethylenically unsaturated double bond with an amine, reaction of compound (C2) having an ethylenically unsaturated double bond with compound (C2) having an amino group, reaction of a compound having an ethylenically unsaturated double bond with compound (C2) having an amino group, reaction of a compound having an acetylenically unsaturated triple bond with compound (C2) having an amino group, reaction of compound (C2) having an imine-based unsaturated double bond with an organic alkali metal compound, reaction of compound (C2) having an imine-based unsaturated double bond with an organic alkaline earth metal compound, and reaction of a carbonyl compound with compound (C2) having an amino group.

<Synthetic Method for Compound Having Ureylene Group as Linking Group and Having Hydrolyzable Silyl Group and/or Silanol Group>

A synthetic method for a compound having an ureylene group (hereinafter, called as appropriate a ‘ureylene linking group-containing C2 compounds’) as a linking group is not particularly limited, but can be synthesized by one of a synthetic method selected from the group comprising reaction of compound (C2) having an amino group with an isocyanate ester, reaction of compound (C2) having an amino group with compound (C2) having an isocyanate ester, and reaction of an amine with compound (C2) having an isocyanate ester.

As the compound (C2) in the present invention, it is preferable to use a silane coupling agent.

### Silane Coupling Agent

The silane coupling agent that is suitable as the compound (C2) in the present invention is explained below.

In the present invention, a functional group in which at least one alkoxy group or halogen group is directly bonded to an Si atom is called a silane coupling group, and a compound containing one or more of the silane coupling groups above in the molecule is called a silane coupling agent. The silane coupling group is preferably one in which two or more alkoxy groups or halogen atoms are directly bonded to an Si atom, and one in which three or more thereof are directly bonded thereto is particularly preferable.

In the resin composition of the present invention, at least one type from a hydrolyzable silyl group and a silanol group in compound (C2), and preferably a silane coupling group in the silane coupling agent, undergoes an alcohol exchange reaction with a reactive functional group in the binder polymer (Component B), for example, if it is a hydroxy group (—OH), with this hydroxy group, thus forming a crosslinked structure. As a result, the binder polymer molecules are three-dimensionally crosslinked via the silane coupling agent. The reactive functional group in the binder polymer (Component B) that can form a crosslinked structure by reacting with at least one type from a hydrolyzable silyl group and a silanol group of compound (C2) is explained in detail below.

The silane coupling agent as a preferred embodiment of the present invention essentially has, as a functional group directly bonded to an Si atom, at least one functional group such as an alkoxy group or a halogen atom, and from the viewpoint of ease of handling of the compound, one having an alkoxy group is preferable.

From the viewpoint of rinsing properties and printing durability, the alkoxy group is preferably an alkoxy group having 1 to 30 carbons, more preferably an alkoxy group having 1 to 15 carbons, and particularly preferably an alkoxy group having 1 to 5 carbons.

Furthermore, examples of the halogen atom include an F atom, a Cl atom, a Br atom, and an I atom, and from the viewpoint of ease of synthesis and stability, a Cl atom and a Br atom are preferable, and a Cl atom is more preferable.

From the viewpoint of maintaining the good balance of the crosslinking level and softness of the film, the silane coupling agent in the present invention contains the silane coupling group preferably at least 1 but no greater than 10 in the molecule, more preferably at least 1 but no greater than 5, and particularly preferably at least 2 but no greater than 4.

When two or more silane coupling groups are contained, preferably the silane coupling groups are linked each other by a linking group. As the linking group, di- or more valent organic groups that may have such substituent as a hetero atom or a hydrocarbon are cited, and, from the viewpoint of a high engraving sensitivity, an embodiment containing a hetero atom (N, S, O) is preferable, and a linking group containing a S atom is particularly preferable.

From such viewpoint, as the silane coupling agent in the present invention, a compound, which has two silane coupling groups having a methoxy group or an ethoxy group, particularly a methoxy group bonded to a Si atom as an alkoxy group in the molecule and these silane coupling groups are bonded via an alkylene group containing a hetero atom (particularly preferably a S atom), is preferable. More specifically, one having a linking group containing a sulfide group is preferable.

Examples of another preferable embodiment of the linking group linking silane coupling groups each other include a linking group having an oxyalkylene group. As the result that the linking group contains an oxyalkylene group, the rinsing properties of engraving residue after the laser engraving is improved. As the oxyalkylene group, an oxyethylene group is preferable, and a polyoxyethylene chain formed by linking plural oxyethylene groups is more preferable. The total number of oxyethylene groups in the polyoxyethylene chain is preferably 2 to 50, more preferably 3 to 30, and particularly preferably 4 to 15.

Specific examples of silane coupling agent can be applied to the present invention are shown below. Examples thereof include b-(3,4-epoxycyclohexyl)ethyltrimethoxysilane, γ-glycidoxypropyltrimethoxysilane, γ-glycidoxypropyldiethoxysilane, γ-glycidoxypropyltriethoxysilane, N-(β-aminoethyl)-γ-aminopropyldimethoxysilane, N-(β-aminoethyl)-γ-aminopropyltrimethoxysilane, N-(β-aminoethyl)-γ-aminopropyltriethoxysilane, γ-aminopropyltrimethoxysilane, γ-aminopropyltriethoxysilane, N-phenyl-γ-aminopropyltrimethoxysilane, γ-mercaptopropyltrimethoxysilane, γ-mercaptopropyltriethoxysilane, bis(triethoxysilylpropyl)disulfide, bis(triethoxysilylpropyl)tetrasulfide, 1,4-bis(triethoxysilyl)benzen, bis(triethoxysilyl)ethane, 1,6-bis(trinmethoxysilyl)hexane, 1,8-bis(triethoxysilyl)octane, 1,2-bis(trimethoxysilyl)decane, bis(triethoxysilylpropyl)amine, bis(trimethoxysilylpropyl)urea, γ-chloropropyltrimethoxysilane, γ-ureidopropyltriethoxysilane, and a compound represented the formulae below is also preferable, but the present invention is not limited to these compounds.

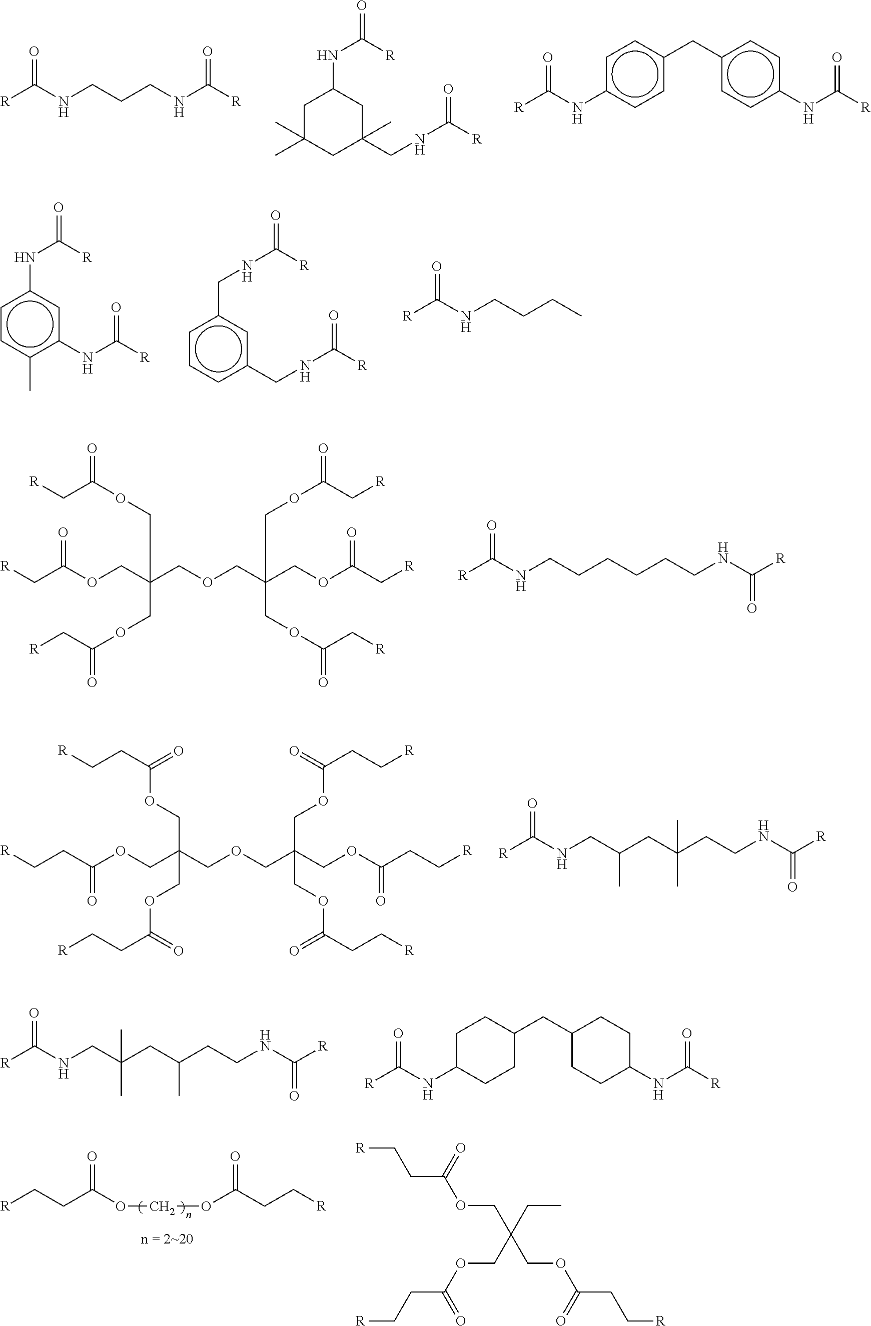

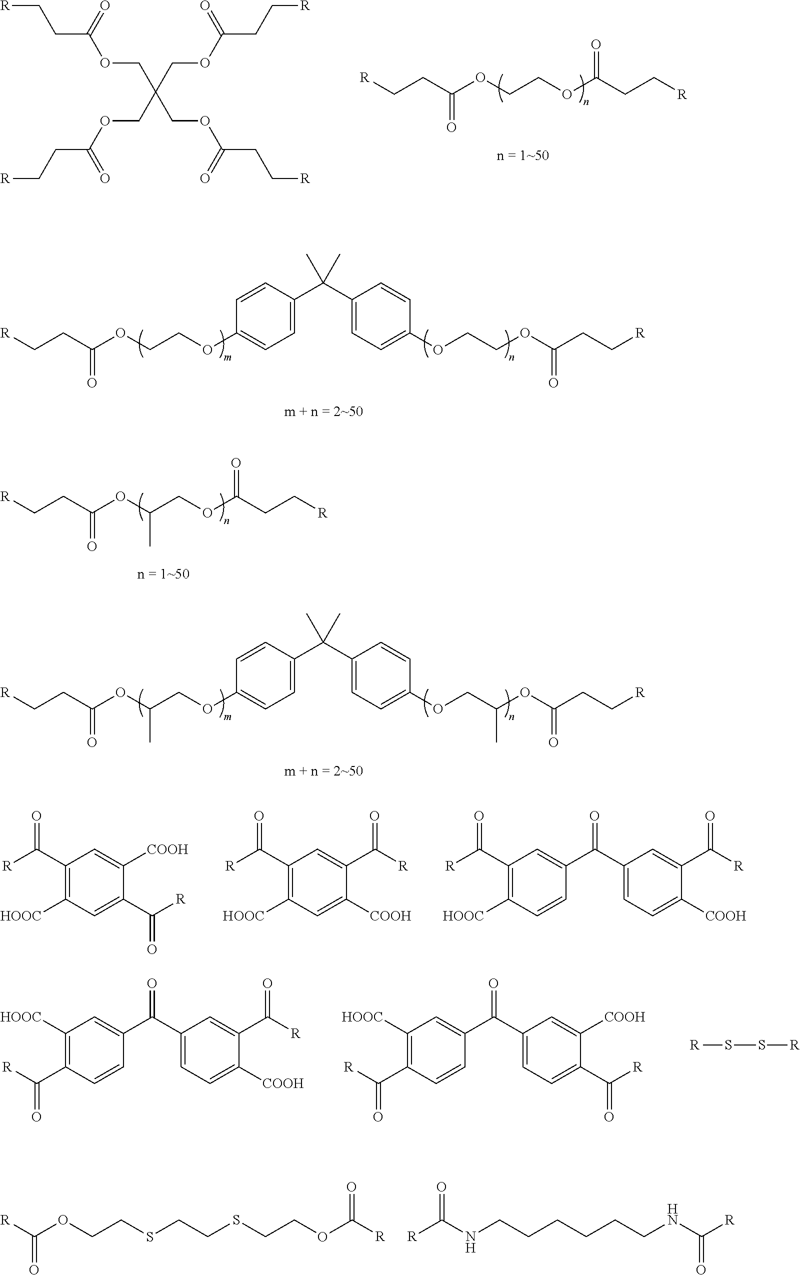

In each of the formulae above, R denotes a partial structure selected from the structures below. When a plurality of Rs and R¹s are present in the molecule, they may be identical to or different from each other, and are preferably identical to each other in terms of synthetic suitability.

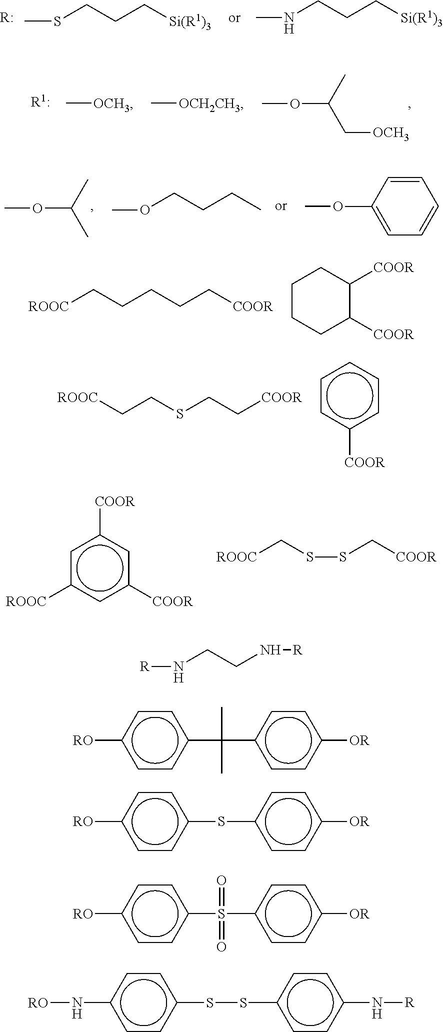

In each of the formulae above, R denotes a partial structure shown below. R1 is the same as defined above. When a plurality of R5 and R1s are present in the molecule, they may be identical to or different from each other, and in terms of synthetic suitability are preferably identical to each other.

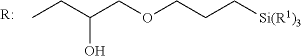

Component A compound may be obtained by synthesis as appropriate, but use of a commercially available product is preferable in terms of cost. Since Component A compound corresponds to for example commercially available silane products or silane coupling agents from Shin-Etsu Chemical Co., Ltd., Dow Corning Toray, Momentive Performance Materials Inc., Chisso Corporation, etc., the resin composition of the present invention may employ such a commercially available product by appropriate selection according to the intended application.

As a silane coupling agent in the present invention, other than the above-mentioned compounds, a partial hydrolysis-condensation product obtained using one type of compound having a hydrolyzable silyl group and/or a silanol group or a partial cohydrolysis-condensation product obtained using two or more types may be used. Hereinafter, these compounds may be called ‘partial (co)hydrolysis-condensation products’.

Specific examples of such partial (co)hydrolysis condensates include a partial (co)hydrolysis condensate obtained by using, as a precursor, one or more selected from the group of silane compounds consisting of alkoxysilane or acetyloxysilane such as tetramethoxysilane, tetraethoxysilane, methyltrimethoxysilane, methyltriethoxysilane, methyltriisopropoxysilane, methyltriacetoxysilane, methyltris(methoxyethoxy)silane, methyltris(methoxypropoxy)silane, ethyltrimethoxysilane, propyltrimethoxysilane, butyltrimethoxysilane, hexyltrimethoxysilane, octyltrimethoxysilane, decyltrimethoxysilane, cyclohexyltrimethoxysilane, phenyltrimethoxysilane, phenyltriethoxysilane, tolyltrimethoxysilane, chloromethyltrimethoxysilane, γ-chloropropyltrimethoxysilane, 3,3,3-trifluoropropyltrimethoxysilane, cyanoethyltriethoxysilane, γ-glycidoxypropyltrimethoxysilane, γ-glycidoxypropyltriethoxysilane, β-(3,4-epoxycyclohexyl)ethyltrimethoxysilane, γ-aminopropyltrimethoxysilane, γ-aminopropyltriethoxysilane, N-β-(aminoethyl)-γ-aminopropyltrimethoxysilane, N-phenyl-γ-aminopropyltrimethoxysilane, γ-mercaptopropyltrimethoxysilane, dimethyldimethoxysilane, dimethyldiethoxysilane, diethyldimethoxysilane, methylethyldimethoxysilane, methylpropyldimethoxysilane, diphenyldimethoxysilane, diphenyldiethoxysilane, methylphenyldimethoxysilane, γ-chloropropylmethyldimethoxysilane, 3,3,3-trifluoropropylmethyldimethoxysilane, γ-glycidoxypropylmethyldiethoxysilane, γ-aminopropylmethyldiethoxysilane, N-β(aminoethyl)-γ-aminopropylmethyldimethoxysilane and γ-mercaptopropylmethyldiethoxysilane, and acyloxysilane such as ethoxalyloxysilane.

Among these silane compounds as partial (co)hydrolysis-condensation product precursors, from the viewpoint of versatility, cost, and film compatibility, a silane compound having a substituent selected from a methyl group and a phenyl group as a substituent on the silicon is preferable, and specific preferred examples of the precursor include methyltrimethoxysilane, methyltriethoxysilane, phenyltrimethoxysilane, phenyltriethoxysilane, dimethyldimethoxysilane, dimethyldiethoxysilane, diphenyldimethoxysilane, and diphenyldiethoxysilane.

In this case, as a partial (co)hydrolysis-condensation product, it is desirable to use a dimer (2 moles of silane compound is reacted with 1 mole of water to eliminate 2 moles of alcohol, thus giving a disiloxane unit) to 100-mer of the above-mentioned silane compound, preferably a dimer to 50-mer, and yet more preferably a dimer to 30-mer, and it is also possible to use a partial cohydrolysis-condensation product formed using two or more types of silane compounds as starting materials.

As such a partial (co)hydrolysis-condensation product, ones commercially available as silicone alkoxy oligomers may be used (e.g. those from Shin-Etsu Chemical Co., Ltd.) or ones that are produced in accordance with a standard method by reacting a hydrolyzable silane compound with less than an equivalent of hydrolytic water and then removing by-products such as alcohol and hydrochloric acid may be used. When the production employs, for example, an acyloxysilane or an alkoxysilane described above as a hydrolyzable silane compound starting material, which is a precursor, partial hydrolysis-condensation may be carried out using as a reaction catalyst an acid such as hydrochloric acid or sulfuric acid, an alkali metal or alkaline earth metal hydroxide such as sodium hydroxide or potassium hydroxide, or an alkaline organic material such as triethylamine, and when the production is carried out directly from a chlorosilane, water and alcohol may be reacted using hydrochloric acid by-product as a catalyst.

With regard to the compound of Component C2 in the resin composition of the present invention, is preferably a silane coupling agent, and it may be used only one type or two or more types in combination.

The total content of Component C contained in the resin composition of the present invention is preferably in the range of 0.1 to 80 wt % on a solids content basis, more preferably in the range of 1 to 40 wt %, and most preferably in the range of 5 to 30 wt %.

In a relief-forming layer related to the present invention, in accordance with the use of a polymerizable compound such as a sulfur-containing polyfunctional monomer, it is possible to adjust film physical properties such as brittleness and flexibility.

Furthermore, from the viewpoint of flexibility or brittleness of a crosslinked film, the total content of (Component C) a polymerizable compound in the relief-forming layer is preferably 5 to 50 weight % on a solids content basis, and more preferably 5 to 30 weight %

When a sulfur-containing polyfunctional monomer is used in combination with other polymerizable compound, the content of sulfur-containing polyfunctional monomer in the total polymerizable compound is preferably at least 5 wt %, and more preferably at least 10 wt %.

### (Component D) Polymerization Initiator

When the resin composition for laser engraving of the present invention is used in production of a relief-forming layer, it preferably further comprises (Component D) a polymerization initiator.

The polymerization initiator (Component D) that can be used in the present invention may be selected from the group of three types of compounds below, which function as crosslinking catalysts. That is, it preferably comprises (Component D-1) an alcohol exchange reaction catalyst, (Component D-2) a polymerization initiator, or (Component D-3) a curing agent that reacts with an epoxy group and/or an oxetanyl group to form a crosslinked structure.

### (Component D-1) Alcohol Exchange Reaction Catalyst

The resin composition for laser engraving of the present invention preferably comprises (Component D-1) an alcohol exchange reaction catalyst in order to promote formation of a crosslinked structure, and more preferably (Component D-1) an alcohol exchange reaction catalyst in combination with a compound containing a hydrolyzable silyl group and/or a silanol group.

With regard to the alcohol exchange reaction catalyst, any reaction catalyst that is usually used in a silane coupling reaction may be used without any limitation.

An acidic catalyst, a basic catalyst, and a metal complex catalyst, which are representative alcohol exchange reaction catalysts, are individually explained below.

### An Acidic or a Basic Catalyst

As the catalyst, an acidic or basic compound is used as it is or in the form of a solution in which it is dissolved in a solvent such as water or an organic solvent (hereinafter, also called an acidic catalyst or basic catalyst respectively). The concentration when dissolved in a solvent is not particularly limited, and it may be selected appropriately according to the properties of the acidic or basic compound used, desired catalyst content, etc.

An acidic or a basic catalyst is not particularly limited. Examples of the acidic catalyst include a hydrogen halide such as hydrochloric acid, nitric acid, sulfuric acid, sulfurous acid, hydrogen sulfide, perchloric acid, hydrogen peroxide, carbonic acid, a carboxylic acid such as formic acid or acetic acid, a carboxylic acid in which R of the structural formula RCOOH is substituted with another element or substituent, a sulfonic acid such as benzenesulfonic acid, phosphoric acid, a heteropoly acid, and an inorganic solid acid.

Examples of the basic catalyst include an ammoniacal base such as aqueous ammonia, an amine such as ethylamine or aniline, an alkali metal hydroxide, an alkali metal alkoxide, an alkaline earth oxide, a quaternary ammonium salt compound, and a quaternary phosphonium salt compound.

As the basic catalyst that can be used in the present invention, an amine is explained as an example below.

Examples of the amine include compounds (a) to (e) shown below.

(a) a nitrogen hydride compound such as hydrazine;
(b) an aliphatic, aromatic or alicyclic, primary, secondary, or tertiary monoamine or polyamine (diamine, triamine, etc.);
(c) a monoamine or polyamine which is a cyclic amine, including a condensed ring, and in which at least one nitrogen atom is contained in the ring skeleton;
(d) an oxygen-containing amine such as an amino acid, an amide, an alcoholamine, an ether amine, an imide, or a lactam;
(e) a hetero element-containing amine containing a heteroatom such as O, S, or Se.

In the case of a secondary or tertiary amine, the substituents on the nitrogen atom (N) may be identical to or different from each other, and among these substituents one or more may be different and the others identical to each other.

Specific examples of the amine include hydrazine,

a primary amine such as monomethylamine, monoethylamine, a monopropylamine, a monobutylamine, a monopentylamine, a monohexylamine, a monoheptylamine, vinylamine, allylamine, a butenylamine, a pentenylamine, a hexenylamine, a pentadienylamine, a hexadienylamine, cyclopentylamine, cyclohexylamine, cyclooctylamine, p-menthylamine, a cyclopentenylamine, a cyclohexenylamine, a cyclohexadienylamine, aniline, benzylamine, naphthylamine, naphthylmethylamine, toluidine, a tolylenediamine, amitrole, ethylenediamine, ethylenetriamine, monoethanolamine, aminothiophene, glycine, alanine, phenylalanine, or aminoacetone,

a secondary amine such as dimethylamine, diethylamine, a dipropylamine, a dibutylamine, a dipentylamine, a dihexylamine, methylethylamine, a methylpropylamine, a methylbutylamine, a methylpentylamine, a methylhexylamine, an ethylpropylamine, an ethylbutylamine, an ethylpentylamine, a propylbutylamine, a propylpentylamine, a propylhexylamine, a butylpentylamine, a pentylhexylamine, divinylamine, diallylamine, a dibutenylamine, a dipentenylamine, a dihexenylamine, methylvinylamine, methylallylamine, a methylbutenylamine, a methylpentenylamine, a methylhexenylamine, ethylvinylamine, ethylallylamine, ethylbutenylamine, an ethylpentenylamine, an ethylhexenylamine, a propylvinylamine, a propylallylamine, a propylbutenylamine, a propylpentenylamine, a propylhexenylamine, a butylvinylamine, a butylallylamine, a butylbutenylamine, a butylpentenylamine, a butylhexenylamine, vinylallylamine, a vinylbutenylamine, a vinylpentenylamine, a vinylhexenylamine, an allylbutenylamine, an allylpentenylamine, an allylhexenylamine, a butenylpentenylamine, a butenylhexenylamine, dicyclopentylamine, dicyclohexyl, methylcyclopentylamine, methylcyclohexylamine, methylcyclooctylamine, ethylcyclopentylamine, ethylcyclohexylamine, ethylcyclooctylamine, a propylcyclopentylamine, a propylcyclohexylamine, a butylcyclopentylamine, a butylcyclohexylamine, a hexylcyclopentylamine, a hexylcyclohexylamine, a hexylcyclooctylamine, vinylcyclopentylamine, vinylcyclohexylamine, vinylcyclooctylamine, allylcyclopentylamine, allylcyclohexylamine, allylcyclooctylamine, a butenylcyclopentylamine, a butenylcyclohexylamine, a butenylcyclooctylamine, a dicyclopentenylamine, a dicyclohexenylamine, a dicyclooctenylamine, a methylcyclopentenylamine, a methylcyclohexenylamine, a methylcyclooctenylamine, an ethylcyclopentenylamine,

an ethylcyclohexenylamine, an ethylcyclooctenylamine, a propylcyclopentenylamine, a propylcyclohexenylamine, a butylcyclopentenylamine, a butylcyclohexenylamine, a vinylcyclopentenylamine, a vinylcyclohexenylamine, a vinylcyclooctenylamine, an allylcyclopentenylamine, an allylcyclohexenylamine, a butenylcyclopentenylamine, a butenylcyclohexenylamine, dicyclopentadienylamine, a dicyclohexadienylamine, a dicyclooctadienylamine, methylcyclopentadienylamine, a methylcyclohexadienylamine, ethylcyclopentadienylamine, an ethylcyclohexadienylamine, a propylcyclopentadienylamine, a propylcyclohexadienylamine, a dicyclooctatrienylamine, a methylcyclooctatrienylamine, an ethylcyclooctatrienylamine, vinylcyclopentadienylamine, a vinylcyclohexadienylamine, allylcyclopentadienylamine, an allylcyclohexadienylamine, diphenylamine, a ditolylamine, dibenzylamine, a dinaphthylamine, N-methylaniline, N-ethylaniline, an N-propylaniline, an N-butylaniline, N-methyltoluidine, N-ethyltoluidine, an N-propyltoluidine, an N-butyltoluidine, N-methylbenzylamine, an N-ethylbenzylamine, an N-propylbenzylamine, an N-butylbenzylamine, an N-methylnaphthylamine, an N-ethylnaphthylamine, an N-propylnaphthylamine, N-vinylaniline, N-allylaniline, N-vinylbenzylamine, N-allylbenzylamine, N-vinyltoluidine, N-allyltoluidine, phenylcyclopentylamine, phenylcyclohexylamine, phenylcyclooctylamine, phenylcyclopentenylamine, phenylcyclohexenylamine, phenylcyclopentadienylamine, N-methylethylenediamine, N,N′-dimethylethylenediamine, N-ethylethylenediamine, N,N′-diethylethylenediamine, an N,N′-dimethyltolylenediamine, an N,N′-diethyltolylenediamine, N-methylethylenetriamine, N,N′-dimethylethylenetriamine, pyrrole, pyrrolidine, imidazole, piperidine, piperazine, a methylpyrrole, a methylpyrrolidine, a methylimidazole, a methylpiperidine, a methylpiperazine, an ethylpyrrole, an ethylpyrrolidine, an ethylimidazole, an ethylpiperidine, an ethylpiperazine, phthalimide, maleimide, caprolactam, pyrrolidone, morpholine, N-methylglycine, N-ethylglycine, N-methylalanine, N-ethylalanine, N-methylaminothiophene, N-ethylaminothiophene, 2,5-piperazinedione, N-methylethanolamine, N-ethylethanolamine, or purine.

Examples of the tertiary amines include trimethylamine, triethylamine, tripropylamines, tributylamines, tripentylamines, trihexylamines, dimethylethylamine, dimethylpropylamines, dimethylbutylamines, dimethylpentylamines, dimethylhexylamines, diethylpropylamines, diethylbutylamines, diethylpentylamines, diethylhexylamines, dipropylbutylamines, dipropylpentylamines, dipropylhexylamines, dibutylpentylamines, dibutylhexylamines, dipentylhexylamines, methyldiethylamine, methyldipropylamines, methyldibutylamines, methyldipentylamines, methyldihexylamines, ethyldipropylamines, ethyldibutylamines, ethyldipentylamines, ethyldihexylamines, propyldibutylamines, propyldipentylamines, propyldihexylamines, butyldipentylamines, butyldihexylamines, pentyldihexylamines, methylethylpropylamines, methylethylbutylamines, methylethylhexylamines, methylpropylbutylamines, methylpropylhexylamines, ethylpropylbutylamine, ethylbutylpentylamines, ethylbutylhexylamines, propylbutylpentylamines, propylbutylhexylamines, butylpentylhexylamines, trivinylamine, triallylamine, tributenylamines, tripentenylamines, trihexenylamines, dimethylvinylamine, dimethylallylamine, dimethylbutenylamines, dimethylpentenylamines, diethylvinylamine, diethylallylamine, diethylbutenylamines, diethylpentenylamines, diethylhexenylamines, dipropylvinylamines, dipropylallylamines, dipropylbutenylamines, methyldivinylamine, methyldiallylamine, methyldibutenylamines, ethyldivinylamine, ethyldiallylamine, tricyclopentylamine, tricyclohexylamine, tricyclooctylamine, tricyclopentenylamine, tricyclohexenylamine, tricyclopentadienylamine, tricyclohexadienylamines, dimethylcyclopentylamine, diethylcyclopentylamine, dipropylcyclopentylamines,

dibutylcyclopentylamines, dimethylcyclohexylamine, diethylcyclohexylamine, dipropylcyclohexylamines, dimethylcyclopentenylamines, diethylcyclopentenylamines, dipropylcyclopentenylamines, dimethylcyclohexenylamines, diethylcyclohexenylamines, dipropylcyclohexenylamines, methyldicyclopentylamine, ethyldicyclopentylamine, propylcyclopentylamines, methyldicyclohexylamine, ethyldicyclohexylamine, propylcyclohexylamines, methyldicyclopentenylamines, ethyldicyclopentenylamines, propyldicyclopentenylamines, N,N-dimethylaniline, N,N-dimethylbenzylamine, N,N-dimethyltoluidines, N,N-dimethylnaphthylamines, N,N-diethylaniline, N,N-diethylbenzylamine, N,N-diethyltoluidines, N,N-diethylnaphthylamines, N,N-dipropylanilines, N,N-dipropylbenzylamines, N,N-dipropyltoluidines, N,N-dipropylnaphthylamines, N,N-divinylaniline, N,N-diallylaniline, N,N-divinyltoluidines, N,N-diallylaniline, diphenylmethylamine, diphenylethylamine, diphenylpropylamines, dibenzylmethylamine, dibenzylethylamine, dibenzylcyclohexylamine, dibenzylvinylamine, dibenzylallylamine, ditolylmethylamines, ditolylethylamines, ditolylcyclohexylamines, ditolylvinylamines, triphenylamine, tribenzylamine, tri(tolyl)amines, trinaphthylamines, N,N,N′,N′-tetramethylethylenediamine, N,N,N′,N′-tetraethylethylenediamine, N,N,N′,N′-tetramethyltolylenediamines, N,N,N′,N′-tetraethyltolylenediamines, N-methylpyrrole, N-methylpyrrolidine, N-ethylimidazole, N,N′-dimethylpiperazine, N-methylpiperidine, N-ethylpyrrole, N-methylpyrrolidine, N-ethylimidazole, N,N′-diethylpiperazine, N-ethylpiperidine, pyridine, pyridazine, pyrazine, quinoline, quinazoline, quinuclidine, N-methylpyrrolidone, N-methylmorpholine, N-ethylpyrrolidone, N-ethylmorpholine, N,N-dimethylanisole, N,N-diethylanisole, N,N-dimethylglycine, N,N-diethylglycine, N,N-dimethylalanine, N,N-diethylalanine, N,N-dimethylethanolamine, N,N-dimethylaminothiophene, 1,1,3,3-tetramethylguanidine, 1,8-diazabicyclo[5.4.0]undeca-7-ene, 1,5-diazabicyclo[4.3.0]nona-5-ene, 1,4-diazabicyclo[2.2.2]octane and hexamethylenetetramine etc.

Therefore, the above-mentioned amine that can be used as the basic catalyst is preferably a compound in which an aliphatic or alicyclic saturated or unsaturated hydrocarbon group, an aromatic hydrocarbon group, an oxygen-containing and/or sulfur-containing and/or selenium-containing hydrocarbon group, etc. is bonded to one or more nitrogen atoms. From the viewpoint of film strength after thermal crosslinking, the pKaH (acid dissociation constant of conjugated acid) range that is preferable as the amine is preferably 7 or greater, and more preferably 10 or greater.

Among the acidic catalysts and basic catalysts, from the viewpoint of an alcohol exchange reaction in the film progressing promptly, methanesulfonic acid, p-toluenesulfonic acid, pyridinium p-toluenesulfonate, dodecylbenzenesulfonic acid, phosphoric acid, phosphonic acid, acetic acid, 1,8-diazabicyclo[5.4.0]undec-7-ene, 1,5-diazabicyclo[4.3.0]non-5-ene, and 1,1,3,3-tetramethylguanidine are preferable, and methanesulfonic acid, p-toluenesulfonic acid, phosphoric acid, 1,8-diazabicyclo[5.4.0]undec-7-ene, and 1,5-diazabicyclo[4.3.0]non-5-ene are particularly preferable.

### <Metal Complex Catalyst>

The metal complex catalyst that can be used as an alcohol exchange reaction catalyst above is preferably constituted from a metal element selected from Groups 2, 4, 5, and 13 of the periodic table and an oxo or hydroxy oxygen compound selected from β-diketones, ketoesters, hydroxycarboxylic acids and esters thereof, amino alcohols, and enolic active hydrogen compounds.

Furthermore, among the constituent metal elements, a Group 2 element such as Mg, Ca, Sr, or Ba, a Group 4 element such as Ti or Zr, a Group 5 element such as V, Nb, or Ta, and a Group 13 element such as Al or Ga are preferable, and they form a complex having an excellent catalytic effect. Among them, a complex obtained from Zr, Al, or Ti is excellent and preferable, ethyl orthotitanate, etc. is more preferable.

In the present invention, examples of the oxo or hydroxy oxygen-containing compound constituting a ligand of the above-mentioned metal complex include β-diketones such as acetylacetone (2,4-pentanedione) and 2,4-heptanedione, ketoesters such as methyl acetoacetate, ethyl acetoacetate, and butyl acetoacetate, hydroxycarboxylic acids and esters thereof such as lactic acid, methyl lactate, salicylic acid, ethyl salicylate, phenyl salicylate, malic acid, tartaric acid, and methyl tartarate, ketoalcohols such as 4-hydroxy-4-methyl-2-pentanone, 4-hydroxy-2-pentanone, 4-hydroxy-4-methyl-2-pentanone, and 4-hydroxy-2-heptanone, amino alcohols such as monoethanolamine, N,N-dimethylethanolamine, N-methylmonoethanolamine, diethanolamine, and triethanolamine, enolic active compounds such as methylolmelamine, methylolurea, methylolacrylamide, and diethyl malonate ester, and compounds having a substituent on the methyl group, methylene group, or carbonyl carbon of acetylacetone.

A preferred ligand is an acetylacetone derivative, and the acetylacetone derivative in the present invention means a compound having a substituent on the methyl group, methylene group, or carbonyl carbon of acetylacetone. The substituent with which the methyl group of acetylacetone is substituted is a straight-chain or branched alkyl group, acyl group, hydroxyalkyl group, carboxyalkyl group, alkoxy group, or alkoxyalkyl group that all have 1 to 3 carbon atoms, the substituent with which the methylene carbon of acetylacetone is substituted is a carboxy group or a straight-chain or branched carboxyalkyl group or hydroxyalkyl group having 1 to 3 carbon atoms, and the substituent with which the carbonyl carbon of acetylacetone is substituted is an alkyl group having 1 to 3 carbon atoms, and in this case the carbonyl oxygen turns into a hydroxy group by addition of a hydrogen atom.

Specific preferred examples of the acetylacetone derivative include acetylacetone, ethyl carbonylacetone, n-propylcarbonylacetone, i-propylcarbonylacetone, diacetylacetone, 1-acetyl-1-propionylacetylacetone, hydroxyethylcarbonylacetone, hydroxypropylcarbonylacetone, acetoacetic acid, acetopropionic acid, diacetoacetic acid, 3,3-diacetopropionic acid, 4,4-diacetobutyric acid, carboxyethylcarbonylacetone, carboxypropylcarbonylacetone, and diacetone alcohol, and among them acetylacetone and diacetylacetone are preferable. The complex of the acetylacetone derivative and the metal element is a mononuclear complex in which 1 to 4 molecules of acetylacetone derivative coordinate to one metal element, and when the number of coordinatable sites of the metal element is larger than the total number of coordinatable bond sites of the acetylacetone derivative, a ligand that is usually used in a normal complex, such as a water molecule, a halide ion, a nitro group, or an ammonio group may coordinate thereto.

Preferred examples of the metal complex include a tris(acetylacetonato)aluminum complex salt, a di(acetylacetonato)aluminum-aqua complex salt, a mono(acetylacetonato)aluminum-chloro complex salt, a di(diacetylacetonato)aluminum complex salt, ethyl acetoacetate aluminum diisopropylate, aluminum tris(ethyl acetoacetate), cyclic aluminum oxide isopropylate, a tris(acetylacetonato)barium complex salt, a di(acetylacetonato)titanium complex salt, a tris(acetylacetonato)titanium complex salt, a di-i-propoxy-bis(acetylacetonato)titanium complex salt, zirconium tris(ethyl acetoacetate), and a zirconium tris(benzoic acid) complex salt. They are excellent in terms of stability in an aqueous coating solution and an effect in promoting gelling in a sol-gel reaction when thermally drying, and among them ethyl acetoacetate aluminum diisopropylate, aluminum tris(ethyl acetoacetate), a di(acetylacetonato)titanium complex salt, and zirconium tris(ethyl acetoacetate) are particularly preferable.

### <(Component D-2) Polymerization Initiator>

The resin composition for laser engraving of the present invention preferably comprises (Component D-2) a polymerization initiator, and more preferably comprises (Component C-1) a polyfunctional ethylenically unsaturated compound and (Component D-2) a polymerization initiator in order to promote formation of the crosslinked structure.

With regard to the polymerization initiator, one known to a person skilled in the art may be used without any limitations. Radical polymerization initiators, which are preferred polymerization initiators, are explained in detail below, but the present invention should not be construed as being limited to these descriptions.

In the present invention, preferred examples of the radical polymerization initiator include (a) an aromatic ketone, (b) an onium salt compound, (c) an organic peroxide, (d) a thio compound, (e) a hexaarylbiimidazole compound, (f) a ketoxime ester compound, (g) a borate compound, (h) an azinium compound, (i) a metallocene compound, (j) an active ester compound, (k) a compound having a carbon halogen bond, and (l) an azo-based compound. Specific examples of the (a) to (l) above are shown below, but the present invention is not limited to these.

In the present invention, (c) an organic peroxide and (l) an azo-based compound is preferable, and (c) an organic peroxide is particurally preferable from the viewpoint of improving the engraving sensitivity and relief edge shape when it is applied to the relief-forming layer in the relief printing plate precursor.

As compound comprises before-mentioned (a) aromatic ketones, (b) onium salt compounds, (d) a thio compound, (e) hexaarylbiimidazole compounds, (f) a ketoxime ester compound, (g) a borate compound, (h) an azinium compound, (i) metallocene compounds, (j) an active ester compound, (k) compounds having a carbon-halogen bond, the compounds described in JP-A-2008-63554, paragraphs 0074 to 0118 can preferably be used.

Examples of (c) organic peroxides and (l) azo-based compounds include compounds as shown below.

### (c) Organic Peroxides

Preferable (c) organic peroxides as the radical polymerization initiator which can be used in the present invention is preferably ether peoxide such as 3,3′,4,4′-tetra(tertiarybutylperoxycarbonyl)benzophenone, 3,3′,4,4′-tetra(tertiaryamylperoxycarbonyl)benzophenone, 3,3′,4,4′-tetra(tertiaryhexylperoxycarbonyl)benzophenone, 3,3′,4,4′-tetra(tertiaryoctylperoxycarbonyl)benzophenone, 3,3′,4,4′-tetra(cumylperoxycarbonyl)benzophenone, 3,3′,4,4′-tetra(p-isopropylcumylperoxycarbonyl)benzophenone, di-tertiarybutyldiperoxy isophthalate etc.

### (l) Azo-Based Compound

Preferable (l) azo-based compounds used as the radical polymerization initiator in the present invention include 2,2′-azobisisobutyronitrile, 2,2′-azobispropionitrile, 1,1′-azobis(cyclohexane-1-carbonitrile), 2,2′-azobis(2-methylbutyronitrile), 2,2′-azobis(2,4-dimethylvaleronitrile), 2,2′-azobis(4-methoxy-2,4-dimethylvaleronitrile), 4,4′-azobis(4-cyanovaleric acid), 2,2′-dimethyl azobisisobutyrate, 2,2′-azobis(2-methylpropionamidoxime), 2,2′-azobis[2-(2-imidazoline-2-yl)propane], 2,2′-azobis{2-methyl-N-[1,1-bis(hydroxymethyl)-2-hydroxyethyl]propionamide}, 2,2′-azobis[2-methyl-N-(2-hydroxyethyl)propionamide], 2,2′-azobis(N-butyl-2-methylpropionamide), 2,2′-azobis(N-cyclohexyl-2-methylpropionamide), 2,2′-azobis[N-(2-propenyl)-2-methylpropionamide], and 2,2′-azobis(2,4,4-trimethylpentane), etc.

In the present invention, the above-mentioned (c) organic peroxide is preferable as the polymerization initiator in the present invention from the viewpoint of the crosslinking properties of the film (relief-forming layer), and particularly preferable from the viewpoint of improving the engraving sensitivity.

From the viewpoint of the engraving sensitivity, an embodiment obtained by combining (c) an organic peroxide and a polymer having a glass transition temperature of ordinary temperature or more, as (Component B) a binder polymer or (Component B-2) a binder polymer for use in combination, is particularly preferable.

This is presumed as follows. When the relief-forming layer is cured by thermal crosslinking using an organic peroxide, an organic peroxide that did not play a part in radical generation and has not reacted remains, and the remaining organic peroxide works as an autoreactive additive and decomposes exothermally in laser engraving. As the result, energy of generated heat is added to the radiated laser energy to thus raise the engraving sensitivity.

It is presumed that, in the case where the glass transition temperature of (Component B) the binder polymer is ordinary temperature or more, the heat generated caused by the decomposition of the organic peroxide is transmitted effectively to (Component B) the binder polymer or (Component B-2) the binder polymer for use in combination and utilized effectively to the thermal decomposition of the binder polymers to thus make the sensitivity more higher.

It will be described in detail in the explanation of a light-heat converting agent, the effect thereof is remarkable when carbon black is used as the light-heat converting agent. It is considered that the heat generated from the carbon black is also transmitted to (c) an organic peroxide and, as the result, heat is generated not only from the carbon black but also from the organic peroxide, and that the generation of heat energy to be used for the decomposition of (Component B) a binder polymer etc. occurs synergistically.

(Component D-3) Curing Agent Capable of Reacting with an Epoxy Group and/or an Oxetanyl Group to Thus Form a Crosslinked Structure

The resin composition for laser engraving of the present invention comprises (Component D-3) a curing agent capable of reacting with an epoxy group and/or an oxetanyl group of Component A to thus form a crosslinked structure.

Since the reaction proceeds rapidly and a film having high strength is obtained, Component D-3 is preferably a compound having one or more functional groups selected from the group consisting of a primary amino group and an acid anhydride group, or a compound having two or more functional groups selected from the group consisting of a secondary amino group, a mercapto group, a carboxyl group, a phenolic hydroxyl group and a hydroxyl group, more preferably a compound having one or more functional groups selected from the group consisting of a primary amino group and an acid anhydride group, or a compound having two or more functional groups selected from the group consisting of a secondary amino group and a mercapto group, and yet more preferably a compound having one or more functional groups selected from the group consisting of a primary amino group and an acid anhydride group.

The compound having at least one primary amino group is not particularly limited, and various types thereof may be used.

Examples thereof include primary alkylamines such as butylamine, octylamine, oleylamine and 2-ethylhexylamine, primary anilines such as aniline, 4-aminoacetophenone, p-anisidine, 2-aminoanthracene and 1-naphthylamine, primary alkanolamines such as monoethanolamine, 2-ethoxyethanolamine and 2-hydroxypropanolamine, aliphatic polyamines such as hexanediamine, ethylenediamine, diethylenetriamine, triethylenetetramine, tetraethylenepentamine, m-xylenediamine and p-xylenediamine, alicyclic polyamines such as 1,3-diaminocyclohexane and isoholondiamine, polyanilines such as 1,4-phenylenediamine, 2,3-diaminonaphthalene, 2,6-diaminoanthraquinone, 2,2-bis(4-aminophenyl)hexafluoropropane, 4,4′-diaminobenzophenone and 4,4′-diaminodiphenylmethane, Mannich bases consisting of a polycondensate of polyamines, an aldehyde compound, mono- or polyvalent phenols, and polyamidopolyamines obtained by the reaction of polyamines with polycarboxylic acid or dimer acid.

Among these, because of the suitability for forming a high degree of three dimensional crosslinking, aliphatic polyamines, alicyclic polyamines and polyanilines are preferable, and, in particular, hexanediamine, triethylenetetramine, m-xylenediamine and 4,4′-diaminodiphenylmethane are more preferable.

The compound having at least two secondary amino groups is not particularly limited, and various types thereof may be used.

Examples thereof include N,N′-dimethylethylenediamine, N,N′-diethylethylenediamine, N,N′-dibenzylethylenediamine, N,N′-diisopropylethylenediamine, 2,5-dimethylpiperazine, N,N′-dimethylcyclohexane-1,2-diamine, piperazine, homopiperazine, 2-methylpiperazine, etc.

The compound having at least one acid anhydride group is not particularly limited, and various types thereof may be used.

Usable examples thereof include acid anhydride compounds such as succinic anhydride, maleic anhydride, phthalic anhydride, hexahydrophthalic anhydride, methylhexahydrophthalic anhydride, nadic anhydride, hydrogenated nadic anhydride, trimellitic anhydride, and pyromellitic anhydride. Among these, the use of methylhexahydrophthalic anhydride is particularly preferable, which gives a cured film that shows a little curing contraction and has transparency and high strength.

The compound having at least two mercapto groups is not particularly limited, and various types thereof may be used.

Examples thereof include alkanedithiols such as 1,2-ethanedithiol, 1,3-propanedithiol, 1,4-butanedithiol, 1,5-pentanedithiol, 1,6-hexanedithiol, 1,7-heptanedithiol, 1,8-octanedithiol, 1,9-nonanedithiol, 1,10-decanedithiol, 1,12-dodecanedithiol, 2,2-dimethyl-1,3-propanedithiol, 3-methyl-1,5-pentanedithiol and 2-methyl-1,8-octanedithiol, cycloalkanedithiols such as 1,4-cyclohexanedithiol, alkanedithiols containing a hetero atom in a carbon chain such as bis(2-mercaptoethyl)ether, bis(2-mercaptoethyl)sulfide, bis(2-mercaptoethyl)disulfide and 2,2′-(ethylenedithio)diethanethiol, alkanedithiols containing a hetero atom and an alicyclic structure in a carbon chain such as 2,5-bis(mercaptomethyl)-1,4-dioxane and 2,5-bis(mercaptomethyl)-1,4-dithiane, alkanetrithiols such as 1,1,1-tris(mercaptomethyl)ethane, 2-ether-2-mercaptomethyl-1,3-propanedithiol and 1,8-mercapto-4-mercaptomethyl-3,6-thiaoctane, alkanetetrathiols such as tetrakis(mercaptomethyl)methane, 3,3′-thiobis(propane-1,2-dithiol), 2,2′-thiobis(propane-1,3-dithiol), etc.

The compound having at least two carboxyl groups is not particularly limited, and various types thereof may be used.

Examples thereof include succinic acid, maleic acid, phthalic acid, hexahydrophthalic acid, methylhexahydrophthalic acid, nadic acid, hydrogenated nadic acid, trimellitic acid, pyromellitic acid, adipic acid, sebacic acid, dodecanedicarboxylic acid, isophthalic acid, 2-methylterephthalic acid, naphthalenedicarboxylic acid, etc.

The compound having at least two phenolic hydroxyl groups is not particularly limited, and various types thereof may be used.

Examples thereof include novolac type resins such as phenolnovolac resin, cresolnovolac resin and naphtholnovolac resin; polyfunctional type phenol resins such as triphenolmethane type resin; modified phenol resins such as dicyclopentanediene-modified phenol resin and terpene-modified phenol resin; aralkyl type resins such as phenolaralkyl resin having a phenylene skeleton, phenolaralkyl resin having a biphenylene skeleton, naphtholaralkyl resin having a phenylene skeleton and naphtholaralkyl resin having a biphenylene skeleton; bisphenol compounds such as bisphenol A and bisphenol F; a sulfur atom-containing type phenol resins such as bisphenol S, etc.

As the compound having at least two hydroxyl groups, various kinds may be used, without particular limitations.

Examples thereof include ethylene glycol, diethylene glycol, propylene glycol, dipropylene glycol, trymethylene glycol, 1,4-tetramethylenediol, 1,3-tetramethylenediol, 2-methyl-1,3-trymethylenediol, 1,5-pentamethylenediol, neopentyl glycol, 1,6-hexamethylenediol, 3-methyl-1,5-pentamethylenediol, 2,4-diethyl-1,5-pentamethylenediol, glycerin, trimethylolpropane, trimethylolethane, cyclohexanediols (such as 1,4-cyclohexanediol), bisphenols (such as bisphenol A), sugar alcohols (such as xylitol and sorbitol), polyalkylene glycols such as polyethylene glycol, polypropylene glycol and polytetramethylene glycol, etc.

With regard to (Component D) a polymerization initiator in the present invention, one type may be used on its own or two or more types may be used in combination.

The content of (Component D) a polymerization initiator in the relief forming layer is preferably 0.01 to 10 weight % relative to the total solids content by weight of the relief-forming layer, and more preferably 0.1 to 3 weight %. When the content of the polymerization initiator is at least 0.01 weight %, an effect from the addition thereof is obtained, and crosslinking of a crosslinkable relief-forming layer proceeds promptly. Furthermore, when the content is no greater than 10 weight %, other components do not become insufficient, and printing durability that is satisfactory as a relief printing plate is obtained.

### (Component E) Plasticizer

The resin composition for laser engraving of the present invention contains preferably a plasticizer. The plasticizer is a material having the function of softening the film formed with the resin composition for laser engraving, and has necessarily a good compatibility relative to the binder polymer.

As the plasticizer, for example, tributyl citrate, dioctyl phthalate, didodecyl phthalate, polyethylene glycols, and polypropylene glycols (such as monool type and diol type) are used preferably.

The resin composition for laser engraving of the present invention may comprise Component E in one kind alone, or in two or more kinds in combination.

The content of Component E in the resin composition for laser engraving of the present invention is, from the viewpoint of lowering the glass transition temperature to room temperature or less, on the solids content basis while defining the total weight of the resin composition as 100 wt %, preferably 1 to 50 wt %, more preferably 10 to 40 wt %, and yet more preferably 20 to 30 wt %.

### <(Component F) Fragrance>

In order to reduce odor, the resin composition for laser engraving of the present invention preferably comprises (Component F) a fragrance. A fragrance is effective in reducing odor when producing a relief printing plate precursor or when carrying out laser engraving.

When the resin composition for laser engraving of the present invention comprises (Component F) a fragrance, the odor of solvent evaporating when drying a liquid-form resin composition coated during production can be masked. Furthermore, unpleasant smell such as amine odor, ketone odor, aldehyde odor, or the foul burning smell of resin occurring when carrying out laser engraving can be masked.

Furthermore, the fragrance is preferably one that not only has an aroma but also can be dissolved well in a compound containing an amino group, a carbonyl group, or an aldehyde group. In order to reduce the smell effectively and function further effectively, the fragrance component preferably has a chemical structure that can be dissolved well in a compound containing an amino group, a carbonyl group, or an aldehyde group. With regard to solubility, a branched chain skeleton is preferred over an open-chain skeleton; among them, when the fragrance is a hydrocarbon compound, an aldehyde compound, a ketone compound, an ester compound, or an alcohol compound, good solubility is exhibited. Furthermore, when it is a cyclic ketone compound or a cyclic ester compound, good solubility is exhibited when the compound has 4 to 20 carbons. Moreover, since a compound having such a structure also has an effect in reducing the smell of sulfur, it is useful for a resin composition containing a compound containing a sulfur atom.

As the fragrance, a known fragrance may be used by appropriate selection; one type of fragrance may be used on its own, or a plurality of fragrances may be used in combination.

The fragrance is preferably selected as appropriate according to the cross linking agent, the polymer, and solvent and it is preferable to carry out optimization by combining known fragrances. Examples of the fragrance include fragrances described in ‘Gosei Koryo—Kagaku To Shohin Chishiki—(Synthetic Fragrances—Chemistry and Product Knowledge—)’ (Motoichi Indo, The Chemical Daily Co., Ltd.), ‘Koryo Kagaku Nyumon (Introduction to Fragrance Chemistry)’ (Shoji Watanabe, Baifukan), ‘Kaori no Hyakka’ (Encyclopedia of Fragrances) (Ed. by Japan Perfumery & Flavoring Association, Asakura Publishing Co., Ltd.), and ‘Koryo Kagaku Soran II (Complete Fragrance Chemistry II) Isolated Fragrances/Synthetic Fragrances/Applications of Fragrances’ (Hirokawa-Shoten Ltd.).

As the fragrance, either a natural fragrance or a synthetic fragrance may be used.

Focusing attention on the molecular structure of the fragrance, examples of the fragrance include hydrocarbons such as an aliphatic hydrocarbon, a terpene hydrocarbon, and an aromatic hydrocarbon, alcohols such as an aliphatic alcohol, a terpene alcohol, and an aromatic alcohol, ethers such as an aliphatic ether and an aromatic ether, oxides such as an aliphatic oxide and a terpene oxide, aldehydes such as an aliphatic aldehyde, a terpene-based aldehyde, an alicyclic aldehyde, a thioaldehyde, and an aromatic aldehyde, ketones such as an aliphatic ketone, a terpene ketone, an alicyclic ketone, an aliphatic cyclic ketone, a non-benzene-based aromatic ketone, and an aromatic ketone, acetals, ketals, phenols, phenol ethers, acids such as a fatty acid, a terpene-based carboxylic acid, an alicyclic carboxylic acid, and an aromatic carboxylic acid, acid amides, lactones such as an aliphatic lactone, a macrocyclic lactone, a terpene-based lactone, an alicyclic lactone, and an aromatic lactone, esters such as an aliphatic ester, a furan-based carboxylic acid ester, an aliphatic cyclic carboxylic acid ester, a cyclohexyl carboxylic acid ester, a terpene-based carboxylic acid ester, and an aromatic carboxylic acid ester, and nitrogen-containing compounds such as a nitro musk, a nitrile, an amine, a pyridine, a quinoline, a pyrrole, and an indole.

Among them, representative examples include acetylcedrene, Iso E Super (brand name), ethyl isovalerate, benzyl isovalerate, ethyl vanillin, ethylene brassylate, 1-octen-3-ol, Galaxolide (brand name), camphor, methyl cinnamate, geraniol, isobornyl acetate, geranyl acetate, benzyl acetate, linalyl acetate, Sandalol (brand name), cyclamenaldehyde, cyclopentadecanolide, citral, citronellal, citronellol, methyl dihydrojasmonate, cis-jasmone, damascone, terpineol, Tonalid (brand name), Bacdanol, vanillin, hydroxycitronellal, phenylacetaldehyde, 2-phenylethanol, hexylcinnamaldehyde, cis-3-hexenol, heliotropin, methyl atrarate, methylionone, menthol, ionone, linalool, Lyral (brand name), Kovanol (brand name), Lilial (brand name), and rose oxide.

Among them, it is preferable to use as the fragrance a terpene compound such as a terpene-based hydrocarbon, a terpene-based alcohol, a terpene oxide, a terpene-based aldehyde, a terpene-based ketone, a terpene-based carboxylic acid, a terpene-based lactone, or a terpene-based carboxylate ester and/or an ester compound such as an aliphatic ester, a furan-based carboxylate ester, an alicyclic carboxylate ester, a cyclohexylcarboxylate ester, a terpene-based carboxylic acid ester, or an aromatic carboxylate ester.

With regard to Component F in the resin composition for laser engraving of the present invention, only one type may be used or two or more types may be used in combination.

The content of (Component F) a fragrance in the resin composition for laser engraving of the present invention is preferably 0.003 to 1.5 wt % relative to the solids contents total weight of the resin composition, and more preferably 0.005 to 1.0 wt %. When in the above-mentioned range, a masking effect can be exhibited fully, the odor of the fragrance is appropriate, the operating environment can be improved, and engraving sensitivity is excellent.

### (Component G) Photothermal Conversion Agent

The resin composition for laser engraving of the present invention preferably further comprises (Component G) a photothermal conversion agent. That is, it is surmised that the photothermal conversion agent in the present invention absorbs laser light and generates heat thus promoting thermal decomposition of a cured material of the resin composition for laser engraving of the present invention. Because of this, it is preferable to select a photothermal conversion agent that absorbs light having the wavelength of the laser that is used for engraving.

When a laser (a YAG laser, a semiconductor laser, a fiber laser, a surface emitting laser, etc.) emitting infrared at a wavelength of 700 to 1,300 nm is used as a light source for laser engraving, it is preferable for the relief-forming layer in the present invention to comprise a photothermal conversion agent that can absorb light having a wavelength of 700 to 1,300 nm.

As the photothermal conversion agent in the present invention, various types of dye or pigment are used.

With regard to the photothermal conversion agent, examples of dyes that can be used include commercial dyes and known dyes described in publications such as ‘Senryo Binran’ (Dye Handbook) (Ed. by The Society of Synthetic Organic Chemistry, Japan, 1970). Specific examples include dyes having a maximum absorption wavelength at 700 to 1,300 nm, such as azo dyes, metal complex salt azo dyes, pyrazolone azo dyes, naphthoquinone dyes, anthraquinone dyes, phthalocyanine dyes, carbonium dyes, diimmonium compounds, quinone imine dyes, methine dyes, cyanine dyes, squarylium dyes, pyrylium salts, and metal thiolate complexes.

In particular, cyanine-based dyes such as heptamethine cyanine dyes, oxonol-based dyes such as pentamethine oxonol dyes, and phthalocyanine-based dyes are preferably used. Examples include dyes described in paragraphs 0124 to 0137 of JP-A-2008-63554.

With regard to the photothermal conversion agent used in the present invention, examples of pigments include commercial pigments and pigments described in the Color Index (C.I.) Handbook, ‘Saishin Ganryo Binran’ (Latest Pigments Handbook) (Ed. by Nippon Ganryo Gijutsu Kyokai, 1977), ‘Saisin Ganryo Ouyogijutsu’ (Latest Applications of Pigment Technology) (CMC Publishing, 1986), ‘Insatsu Inki Gijutsu’ (Printing Ink Technology) CMC Publishing, 1984).

Examples of the type of pigment include black pigments, yellow pigments, orange pigments, brown pigments, red pigments, violet pigments, blue pigments, green pigments, fluorescent pigments, metal powder pigments, and other polymer-bonding colorants. Specific examples include insoluble azo pigments, azo lake pigments, condensed azo pigments, chelate azo pigments, phthalocyanine-based pigments, anthraquinone-based pigments, perylene and perinone-based pigments, thioindigo-based pigments, quinacridone-based pigments, dioxazine-based pigments, isoindolinone-based pigments, quinophthalone-based pigments, dyed lake pigments, azine pigments, nitroso pigments, nitro pigments, natural pigments, fluorescent pigments, inorganic pigments, and carbon black. Among these pigments, carbon black is preferable.

Any carbon black, regardless of classification by ASTM and application (e.g. for coloring, for rubber, for dry cell, etc.), may be used as long as dispersibility, etc. in the composition is stable. Carbon black includes for example furnace black, thermal black, channel black, lamp black, and acetylene black. In order to make dispersion easy, a black colorant such as carbon black may be used as color chips or a color paste by dispersing it in nitrocellulose or a binder in advance using, as necessary, a dispersant, and such chips and paste are readily available as commercial products.

In the present invention, it is possible to use carbon black having a relatively low specific surface area and a relatively low dibutyl phthalate (DBP) absorption and also finely divided carbon black having a large specific surface area. Preferred examples of carbon black include Printex (registered trademark) U, Printex (registered trademark) A, and Spezialschwarz (registered trademark) 4 (Degussa).

From the viewpoint of improving engraving sensitivity by efficiently transmitting heat generated by photothermal conversion to the surrounding polymer, etc., the carbon black that can be applicable in the present invention is preferably a conductive carbon black having a specific surface area of at least 150 m²/g and a DBP number of at least 150 mL/100 g.

This specific surface area is preferably at least 250 m²/g, and particularly preferably at least 500 m²/g. The DBP number is preferably at least 200 mL/100 g, and particularly preferably at least 250 mL/100 g. The above-mentioned carbon black may be acidic or basic carbon black. The carbon black is preferably basic carbon black. It is of course possible to use a mixture of different carbon blacks.

Conductive carbon black having a specific surface area of up to about 1,500 m²/g and a DBP number of up to about 550 mL/100 g is commercially available under names such as for example Ketjenblack (registered trademark) EC300J, Ketjenblack (registered trademark) EC600J (Akzo), Printex (registered trademark) XE (Degussa), Black Pearls (registered trademark) 2000 (Cabot), and Ketjen Black (Lion Corporation).

When carbon black is used as the photothermal conversion agent, thermal crosslinking is more preferable in point of the curability of the film, instead of the photo crosslinking using UV light etc., and, by the combination with (c) the organic peroxide as (Component B) the polymerization initiator, which is the aforementioned preferable component for use in combination, the engraving sensitivity becomes extremely high, more preferably.

As the most preferable embodiment of the present invention, as described above, an embodiment, in which (Component B) a binder polymer, (c) an organic peroxide as (Component D) a polymerization initiator and carbon black as (Component G) a photothermal conversion agent are used in combination, can be cited.

The content of the photothermal conversion agent in the resin composition for laser engraving of the present invention largely depends on the size of the molecular extinction coefficient characteristic to the molecule, and is preferably 0.01 to 20 wt % relative to the solids content total weight of the resin composition, more preferably 0.05 to 10 wt %, and yet more preferably 0.1 to 5 wt %.

### <Solvent>

An aprotic organic solvent which solves Component A, etc. constituting the resin composition is preferably used as a major component when preparing the resin composition for laser engraving of the present invention. More specifically, they are used preferably at ratio of aprotic organic solvent/protic organic solvent=100/0 to 50/50 (ratio by weight), more preferably 100/0 to 70/30, and particularly preferably 100/0 to 90/10.

Specific preferred examples of the aprotic organic solvent include acetonitrile, tetrahydrofuran, dioxane, toluene, propylene glycol monomethyl ether acetate, methyl ethyl ketone, acetone, methyl isobutyl ketone, ethyl acetate, butyl acetate, ethyl lactate, γ-butyrolactone, N,N-dimethylacetamide, N-methylpyrrolidone, and dimethyl sulfoxide.

Specific preferred examples of the protic organic solvent include methanol, ethanol, 1-propanol, 2-propanol, 1-butanol, 1-methoxy-2-propanol, ethylene glycol, diethylene glycol, and 1,3-propanediol.

It is preferable to use only γ-butyrolactone and more preferable to use 10-40 weight parts relative to 100 weight parts of the total solid contents in the resin composition.

### Other Additives

The resin composition for laser engraving of the present invention preferably comprises, as an additive for improving engraving sensitivity, nitrocellulose or a high thermal conductivity material. Since nitrocellulose is a self-reactive compound, it generates heat during laser engraving, thus assisting thermal decomposition of a coexisting binder polymer such as a hydrophilic polymer. It is surmised that as a result, the engraving sensitivity improves. A high thermal conductivity material is added for the purpose of assisting heat transfer, and examples of thermally conductive materials include inorganic compounds such as metal particles and organic compounds such as a conductive polymer. As the metal particles, fine gold particles, fine silver particles, and fine copper particles having a particle diameter of on the order of a micrometer or a few nanometers are preferable. As the conductive polymer, a conjugated polymer is particularly preferable, and specific examples thereof include polyaniline and polythiophene.

Furthermore, use of a co-sensitizer enables the sensitivity to be improved when the resin composition for laser engraving is photocured.

Moreover, in order to prevent unwanted thermal polymerization of a polymerizable compound during production or storage of the composition, a small amount of thermopolymerization inhibitor is desirably added.

For the purpose of coloring the resin composition for laser engraving, a colorant such as a dye or a pigment may be added. This enables properties such as visibility of an image area or suitability for an image densitometer to be improved.

Furthermore, in order to improve the physical properties of a cured film of the resin composition for laser engraving, a known additive such as a filler may be added.

### (Relief Printing Plate Precursor for Laser Engraving)

A first embodiment of the relief printing plate precursor for laser engraving of the present invention comprises a relief-forming layer formed from the resin composition for laser engraving of the present invention.

A second embodiment of the relief printing plate precursor for laser engraving of the present invention comprises a crosslinked relief-forming layer formed by crosslinking a relief-forming layer formed from the resin composition for laser engraving of the present invention.

In the present invention, the ‘relief printing plate precursor for laser engraving’ means both or one of a plate having a crosslinkable relief-forming layer formed from the resin composition for laser engraving in a state before being crosslinked and a plate in a state in which it is cured by light and/or heat.

In the present invention, the ‘relief-forming layer’ means a layer in a state before being crosslinked, that is, a layer formed from the resin composition for laser engraving of the present invention, which may be dried as necessary.

The ‘relief printing plate’ is prepared by laser engraving a printing plate precursor having a crosslinked relief-forming layer.

In the present invention, the ‘crosslinked relief-forming layer’ means a layer formed by crosslinking the relief-forming layer. The crosslinking is preferably carried out by means of heat and/or light. Furthermore, the crosslinking is not particularly limited as long as it is a reaction by which the resin composition is cured, and crosslinked structure is exemplified by due to reactions between Component B's, or between Component B and other Components.

Moreover, in the present invention, the ‘relief layer’ means a layer of the relief printing plate formed by engraving using a laser, that is, the crosslinked relief-forming layer after laser engraving.

A relief printing plate precursor for laser engraving of the present invention comprises a relief-forming layer formed from the resin composition for laser engraving of the present invention, which has the above-mentioned components. The (crosslinked) relief-forming layer is preferably provided above a support.

The (crosslinked) relief printing plate precursor for laser engraving may further comprise, as necessary, an adhesive layer between the support and the (crosslinked) relief-forming layer and, above the relief-forming layer, a slip coat layer and a protection film.

### <Relief-Forming Layer>

The relief-forming layer is a layer formed from the resin composition for laser engraving of the present invention, and is preferably crosslinkable by heat.

As a mode in which a relief printing plate is prepared using the relief printing plate precursor for laser engraving, a mode in which a relief printing plate is prepared by crosslinking a relief-forming layer to thus form a relief printing plate precursor having a crosslinked relief-forming layer, and the crosslinked relief-forming layer (hard relief-forming layer) is then laser-engraved to thus form a relief layer is preferable. By crosslinking the relief-forming layer, it is possible to prevent abrasion of the relief layer during printing, and it is possible to obtain a relief printing plate having a relief layer with a sharp shape after laser engraving.

The relief-forming layer may be formed by molding the resin composition for laser engraving that has the above-mentioned components for a relief-forming layer into a sheet shape or a sleeve shape. The relief-forming layer is usually provided above a support, which is described later, but it may be formed directly on the surface of a member such as a cylinder of equipment for plate producing or printing or may be placed and immobilized thereon, and a support is not always required.

A case in which the relief-forming layer is mainly formed in a sheet shape is explained as an Example below.

### <Support>

A material used for the support of the relief printing plate precursor for laser engraving is not particularly limited, but one having high dimensional stability is preferably used, and examples thereof include metals such as steel, stainless steel, or aluminum, plastic resins such as a polyester (e.g. polyethylene terephthalate (PET), polybutylene terephthalate (PBT), or polyacrylonitrile (PAN)) or polyvinyl chloride, synthetic rubbers such as styrene-butadiene rubber, and glass fiber-reinforced plastic resins (epoxy resin, phenolic resin, etc.). As the support, a PET film or a steel substrate is preferably used. The configuration of the support depends on whether the relief-forming layer is in a sheet shape or a sleeve shape.

### Adhesive Layer

When the relief-forming layer is formed above a support, an adhesive layer may be provided between the two for the purpose of strengthening the adhesive power between the layers.

Any material may be used in the adhesive layer as long as the adhesive power is strengthened after the relief-forming layer is crosslinked, and it is preferable for the adhesive power to be strong before the relief-forming layer is crosslinked. Here, the adhesive power means the adhesive power both between the support/adhesive layer and between the adhesive layer/relief-forming layer.

The adhesive power between the support/adhesive layer is preferably such that, when stripping the adhesive layer and the relief-forming layer from a laminate formed from the support/adhesive layer/relief-forming layer at a speed of 400 mm/min, the peeling force per cm width of the sample is at least 1.0 N/cm or peeling is impossible, and is more preferably at least 3.0 N/cm or peeling is impossible.

The adhesive power between the adhesive layer/relief-forming layer is preferably such that, when stripping the adhesive layer from the adhesive layer/relief-forming layer at a speed of 400 mm/min, the peeling force per cm width of the sample is at least 1.0 N/cm or peeling is impossible, and is more preferably at least 3.0 N/cm or peeling is impossible.

Examples of materials (adhesives) that can be used in the adhesive layer include those described in ‘Handbook of Adhesives’, Second Edition, Ed by I. Skeist (1977).

### Protection Film and Slip Coat Layer

The relief-forming layer becomes a part (relief layer) where a relief is shaped after laser engraving, and the relief layer surface functions as an ink laydown part. Since the crosslinked relief-forming layer is strengthened by crosslinking, hardly any scratches or dents that would affect printing occur on the relief-forming layer surface. However, the strength of the relief-forming layer prior to crosslinking is often insufficient, and the surface is susceptible to scratches or dents. From such a viewpoint, for the purpose of preventing scratches or dents in the relief-forming layer surface, a protection film may be provided on the relief-forming layer surface.

When the protection film is too thin, an effect in preventing scratches or dents cannot be obtained, and when it is too thick, handling becomes difficult and the cost becomes high. The thickness of the protection film is preferably 25 to 500 μm, and more preferably 50 to 200 μm.

The protection film may employ a material known as a protection film for a printing plate, for example, a polyester-based film such as PET (polyethylene terephthalate) or a polyolefin-based film such as PE (polyethylene) or PP (polypropylene). The surface of the film may be plain or made matte.

When a protection film is provided above the relief-forming layer, the protection film should be peelable.

When the protection film is not peelable or conversely has poor adhesion to the relief-forming layer, a slip coat layer may be provided between the two layers. The material used in the slip coat layer preferably employs as a main component a resin that is soluble or dispersible in water and has little tackiness, such as polyvinyl alcohol, polyvinyl acetate, partially saponified polyvinyl alcohol, a hydroxyalkylcellulose, an alkylcellulose, or a polyamide resin.

The material used in the slip coat layer preferably employs as a main component a resin that is soluble or dispersible in water and has little tackiness, such as polyvinyl alcohol, polyvinyl acetate, partially saponified polyvinyl alcohol, a hydroxyalkylcellulose, an alkylcellulose, or a polyamide resin. Among them, from the viewpoint of tackiness, it is particularly preferable for a partially saponified polyvinyl alcohol having a degree of saponification of 60 to 99 mol %, or a hydroxyalkylcellulose or alkylcellulose with an alkyl group having 1 to 5 carbons to be used.

When the protection film is stripped from the relief-forming layer (and slip coat layer)/protection film at a speed of 200 mm/min, the peeling force per cm is preferably 5 to 200 mN/cm, and more preferably 10 to 150 mN/cm. When it is at least 5 mN/cm, the protection film is not stripped during operation, and when it is no greater than 200 mN/cm, the protection film can be stripped without difficulty.

### <Process for Producing Relief Printing Plate Precursor for Laser Engraving>

Process for producing relief printing plate precursor for laser engraving is not particularly limited, and examples thereof include a method in which a resin composition for laser engraving is prepared, solvent is removed from this coating solution composition for laser engraving, and it is then melt-extruded onto a support. Alternatively, a method may be employed in which a resin composition for laser engraving is cast onto a support, and this is dried in an oven to thus remove solvent from the resin composition.

Among them, the process for producing a relief printing plate precursor for laser engraving of the present invention is preferably a production process comprising a layer formation step of forming a relief-forming layer from the resin composition for laser engraving of the present invention and a crosslinking step of crosslinking the relief-forming layer by means of heat and/or light to thus obtain a relief printing plate precursor having a crosslinked relief-forming layer.

Subsequently, as necessary, a protection film may be laminated on the relief-forming layer. Laminating may be carried out by compression-bonding the protection film and the relief-forming layer by means of heated calendar rollers, etc. or putting a protection film into intimate contact with a relief-forming layer whose surface is impregnated with a small amount of solvent.

When a protection film is used, a method in which a relief-forming layer is first layered on a protection film and a support is then laminated may be employed.

When an adhesive layer is provided, it may be dealt with by use of a support coated with an adhesive layer. When a slip coat layer is provided, it may be dealt with by use of a protection film coated with a slip coat layer.

### <Layer Formation Step>

The process for producing the relief printing plate precursor for laser engraving of the present invention preferably comprises a layer formation step of forming a relief-forming layer from the resin composition for laser engraving of the present invention.

Preferred examples of a method for forming the relief-forming layer include a method in which the resin composition for laser engraving of the present invention is prepared, solvent is removed as necessary from this resin composition for laser engraving, and it is then melt-extruded onto a support and a method in which the resin composition for laser engraving of the present invention is prepared, the resin composition for laser engraving of the present invention is cast onto a support, and this is dried in an oven to thus remove solvent.

The resin composition for laser engraving may be produced by, for example, dissolving or dispersing Component A or further any one of Components B to F as optional components in an appropriate solvent, and then mixing these solutions. It is preferable to remove almost all of the solvent component in a stage of producing a relief printing plate precursor. It is therefore preferable to use as the solvent a volatile one such as low molecular weight alcohol (for example, methanol, ethanol, n-propanol, isopropanol, propylene glycol monomethylether) and adjust the temperature of solution, etc. to thus reduce as much as possible the total amount of solvent to be added.

The thickness of the (crosslinked) relief-forming layer in the relief printing plate precursor for laser engraving is preferably 0.05 to 10 mm before and after crosslinking, more preferably 0.05 to 7 mm, and yet more preferably 0.05 to 3 mm.

### <Crosslinking Step>

The process for producing a relief printing plate precursor for laser engraving of the present invention is preferably a production process comprising a crosslinking step of crosslinking the relief-forming layer by means of heat and/or light to thus obtain a relief printing plate precursor having a crosslinked relief-forming layer, and is more preferably a production process comprising a crosslinking step of crosslinking the relief-forming layer by means of heat to thus obtain a relief printing plate precursor having a crosslinked relief-forming layer.

The relief-forming layer may be crosslinked by heating the relief printing plate precursor for laser engraving (step of crosslinking by means of heat). As heating means for carrying out crosslinking by heat, there can be cited a method in which a printing plate precursor is heated in a hot air oven or a far-infrared oven for a predetermined period of time and a method in which it is put into contact with a heated roller for a predetermined period of time.

Due to the relief-forming layer being thermally crosslinked, firstly, a relief formed after laser engraving becomes sharp and, secondly, tackiness of engraving residue formed when laser engraving is suppressed.

Furthermore, a photopolymerization initiator, etc. may be used, and in order to polymerize a polymerizable compound to form crosslinking, crosslinking may further be carried out by light.

When the relief-forming layer contains a photopolymerization initiator, the relief-forming layer may be crosslinked by irradiating the relief-forming layer with actinic radiation that functions as a trigger for the photopolymerization initiator.

With regard to irradiation with light, it is usually carried out for the entire surface of the relief-forming layer. Examples of the light (also called ‘actinic radiation’) include visible light, UV light, and an electron beam, and UV light is most commonly used. When a side where a substrate for immobilizing the relief-forming layer such as the support for the relief-forming layer is present is defined as the reverse face, only the front face need be irradiated with light, but when the support is a transparent film through which actinic radiation passes, it is preferable to further irradiate the reverse face with light as well. When a protection film is present, irradiation from the front face may be carried out with the protection film as it is or after peeling off the protection film. Since there is a possibility of a polymerization reaction being inhibited in the presence of oxygen, irradiation with actinic radiation may be carried out after superimposing a vinyl chloride sheet on the relief-forming layer and evacuating.

### (Relief Printing Plate and Process for Producing Same)

The process for producing a relief printing plate of the present invention preferably comprises a layer formation step of forming a relief-forming layer from the resin composition for laser engraving of the present invention, a crosslinking step of crosslinking the relief-forming layer by means of heat and/or light to thus obtain a relief printing plate precursor having a crosslinked relief-forming layer, and an engraving step of laser-engraving the relief printing plate orecursor having the crosslinked relief-forming layer.

The relief printing plate of the present invention is a relief printing plate having a relief layer obtained by crosslinking and laser-engraving a layer formed from the resin composition for laser engraving of the present invention, and is preferably a relief printing plate made by the process for producing a relief printing plate of the present invention.

The relief printing plate of the present invention may suitably employ an aqueous ink when printing.

The layer formation step and the crosslinking step in the process for producing a relief printing plate of the present invention mean the same as the layer formation step and the crosslinking step in the above-mentioned process for producing a relief printing plate precursor for laser engraving, and preferred ranges are also the same.

### <Engraving Step>

The process for producing a relief printing plate of the present invention preferably comprises an engraving step of laser-engraving the relief printing plate precursor having a crosslinked relief-forming layer.

The engraving step is a step of laser-engraving a crosslinked relief-forming layer that has been crosslinked in the crosslinking step to thus form a relief layer. Specifically, it is preferable to engrave a crosslinked relief-forming layer that has been crosslinked by irradiation with laser light according to a desired image, thus forming a relief layer. Furthermore, a step in which a crosslinked relief-forming layer is subjected to scanning irradiation by controlling a laser head using a computer in accordance with digital data of a desired image can preferably be cited.

This engraving step preferably employs an infrared laser. When irradiated with an infrared laser, molecules in the crosslinked relief-forming layer undergo molecular vibration, thus generating heat. When a high power laser such as a carbon dioxide laser or a YAG laser is used as the infrared laser, a large quantity of heat is generated in the laser-irradiated area, and molecules in the crosslinked relief-forming layer undergo molecular scission or ionization, thus being selectively removed, that is, engraved. The advantage of laser engraving is that, since the depth of engraving can be set freely, it is possible to control the structure three-dimensionally. For example, for an area where fine halftone dots are printed, carrying out engraving shallowly or with a shoulder prevents the relief from collapsing due to printing pressure, and for a groove area where a fine outline character is printed, carrying out engraving deeply makes it difficult for ink the groove to be blocked with ink, thus enabling breakup of an outline character to be suppressed.

In particular, when engraving is carried out using an infrared laser that corresponds to the absorption wavelength of the photothermal conversion agent, it becomes possible to selectively remove the crosslinked relief-forming layer at higher sensitivity, thus giving a relief layer having a sharp image.

As the infrared ray laser used in the engraving step, from the viewpoint of productivity, cost, etc., a carbon dioxide laser or a semiconductor laser is preferable. In particular, a fiber-coupled semiconductor infrared laser is preferably used. In general, compared with a CO2 laser, a semiconductor laser has higher efficiency laser oscillation, is less expensive, and can be made smaller. Furthermore, it is easy to form an array due to the small size. Moreover, the shape of the beam can be controlled by treatment of the fiber.

With regard to the semiconductor laser, one having a wavelength of 700 to 1,300 nm is preferable, one having a wavelength of 800 to 1,200 nm is more preferable, one having a wavelength of 860 to 1,200 nm is further preferable, and one having a wavelength of 900 to 1,100 nm is particularly preferable.

Furthermore, the fiber-coupled semiconductor laser can output laser light efficiently by being equipped with optical fiber, and this is effective in the engraving step in the present invention. Moreover, the shape of the beam can be controlled by treatment of the fiber. For example, the beam profile may be a top hat shape, and energy can be applied stably to the plate face. Details of semiconductor lasers are described in ‘Laser Handbook 2^{nd} Edition’ The Laser Society of Japan, ‘Applied Laser Technology’, The Institute of Electronics and Communication Engineers, etc.

Moreover, as plate producing equipment comprising a fiber-coupled semiconductor laser that can be used suitably in the process for producing a relief printing plate employing the relief printing plate precursor of the present invention, those described in detail in JP-A-2009-172658 and JP-A-2009-214334 can be cited. Such equipment comprising a fiber-coupled semiconductor laser can be used to produce a relief printing plate of the present invention.

The process for producing a relief printing plate of the present invention may as necessary further comprise, subsequent to the engraving step, a rinsing step, a drying step, and/or a post-crosslinking step, which are shown below.

Rinsing step: a step of rinsing the engraved surface by rinsing the engraved relief layer surface with water or a liquid comprising water as a main component.

Drying step: a step of drying the engraved relief layer.

Post-crosslinking step: a step of further crosslinking the relief layer by applying energy to the engraved relief layer.

After the above-mentioned step, since engraving residue is attached to the engraved surface, a rinsing step of washing off engraving residue by rinsing the engraved surface with water or a liquid comprising water as a main component may be added. Examples of rinsing means include a method in which washing is carried out with tap water, a method in which high pressure water is spray-jetted, and a method in which the engraved surface is brushed in the presence of mainly water using a batch or conveyor brush type washout machine known as a photosensitive resin letterpress plate processor, and when slime due to engraving residue cannot be eliminated, a rinsing liquid to which a soap or a surfactant is added may be used.

When the rinsing step of rinsing the engraved surface is carried out, it is preferable to add a drying step of drying an engraved relief-forming layer so as to evaporate rinsing liquid.

Furthermore, as necessary, a post-crosslinking step for further crosslinking the relief-forming layer may be added. By carrying out a post-crosslinking step, which is an additional crosslinking step, it is possible to further strengthen the relief formed by engraving.

The pH of the rinsing liquid that can be used in the present invention is preferably at least 9, more preferably at least 10, and yet more preferably at least 11. The pH of the rinsing liquid is preferably no greater than 14, more preferably no greater than 13.5, and yet more preferably no greater than 13.2, and especially preferably no greater than 12.5. When in the above-mentioned range, handling is easy.

In order to set the pH of the rinsing liquid in the above-mentioned range, the pH may be adjusted using an acid and/or a base as appropriate, and the acid or base used is not particularly limited.

The rinsing liquid that can be used in the present invention preferably comprises water as a main component.

The rinsing liquid may contain as a solvent other than water a water-miscible solvent such as an alcohol, acetone, or tetrahydrofuran.

The rinsing liquid preferably comprises a surfactant.

From the viewpoint of removability of engraving residue and little influence on a relief printing plate, preferred examples of the surfactant that can be used in the present invention include betaine compounds (amphoteric surfactants) such as a carboxybetaine compound, a sulfobetaine compound, a phosphobetaine compound, an amine oxide compound, and a phosphine oxide compound.

Furthermore, examples of the surfactant also include known anionic surfactants, cationic surfactants, and nonionic surfactants. Moreover, a fluorine-based or silicone-based nonionic surfactant may also be used in the same manner.

With regard to the surfactant, one type may be used on its own or two or more types may be used in combination.

It is not necessary to particularly limit the amount of surfactant used, but it is preferably 0.01 to 20 wt % relative to the total weight of the rinsing liquid, and more preferably 0.05 to 10 wt %.

The relief printing plate of the present invention having a relief layer may be produced as described above.

From the viewpoint of satisfying suitability for various aspects of flexographic printing, such as abrasion resistance and ink transfer properties, the thickness of the relief layer of the relief printing plate is preferably at least 0.05 mm but no greater than 10 mm, more preferably at least 0.05 mm but no greater than 7 mm, and yet more preferably at least 0.05 mm but no greater than 0.3 mm.

Furthermore, the Shore A hardness of the relief layer of the relief printing plate is preferably at least 50° but no greater than 90°. When the Shore A hardness of the relief layer is at least 50°, even if fine halftone dots formed by engraving receive a strong printing pressure from a letterpress printer, they do not collapse and close up, and normal printing can be carried out. Furthermore, when the Shore A hardness of the relief layer is no greater than 90°, even for flexographic printing with kiss touch printing pressure it is possible to prevent patchy printing in a solid printed part.

The Shore A hardness in the present specification is a value measured by a durometer (a spring type rubber hardness meter) that presses an indenter (called a pressing needle or indenter) into the surface of a measurement target at 25° C. so as to deform it, measures the amount of deformation (indentation depth), and converts it into a numerical value.

The relief printing plate of the present invention is particularly suitable for printing by a flexographic printer using an aqueous ink, but printing is also possible when it is carried out by a letterpress printer using any of aqueous, oil-based, and UV inks, and printing is also possible when it is carried out by a flexographic printer using a UV ink. The relief printing plate of the present invention has excellent rinsing properties, there is no engraving residue, since a relief layer obtained has excellent elasticity aqueous ink transfer properties and printing durability are excellent, and printing can be carried out for a long period of time without plastic deformation of the relief layer or degradation of printing durability.

In accordance with the present invention, there can be provided a resin composition for laser engraving that is suitable for a relief-forming layer of a relief printing plate precursor for laser engraving, the relief-forming layer having excellent rinsing properties with respect to engraving residue formed by laser engraving and being suitably applicable to an aqueous ink and/or a UV ink during printing.

Furthermore, in accordance with the present invention, due to the use of the resin composition for laser engraving of the present invention, there can be provided a relief printing plate precursor that enables a relief layer having excellent resistance toward both an aqueous ink and a UV ink to be formed, a process for producing the precursor, and a process for making a relief printing plate.

### EXAMPLES

The present invention is explained in further detail below by reference to Examples, but the present invention should not be construed as being limited to these Examples. Part means part by weight unless otherwise specified.

The weight-average molecular weight (Mw) of a polymer in the Examples is a value measured by a GPC method unless otherwise specified.

### Example 1

### 1. Preparation of Crosslinkable Resin Composition for Laser Engraving

A three-necked flask equipped with a stirring blade and a condenser was charged with 30 parts of B-1 ‘Denka Butyral #3000-2’ (Denki Kagaku Kogyo Kabushiki Kaisha, polyvinylbutyral derivative Mw=90,000) as (Component B) binder polymer and 20 parts of γ-butyrolactone as a solvent, and heated at 70° C. for 120 minutes while stirring to thus dissolve the polymer. Subsequently, the solution was set at 40° C., and 10 parts of A-1 as Component A, 25 parts of monomer (C-1) glycerol dimethacrylate as Component C (polymerizable compound), 30 parts of E-1 tributyl citrate as Component E (plasticizer), 1 part of F-1 vanillin (Wako Pure Chemical Industries, Ltd.) as Component F, and 1 part of G-1 Ketjen Black EC600JD (carbon black, Lion Corporation) as Component G (photothermal conversion agent) were further added, and stirring was carried out for 30 minutes. Subsequently, 10 parts of A-1: allyl[3-(2-nitrobenzenesulfonamido)propyl]carbamate (Tokyo Chemical Industry Co., Ltd.) as Component A and 1 part of D-1 dialkyl peroxide (product name: Percumyl, NOF Corporation) as Component D (polymerization initiator) were added, and stirring was carried out at 40° C. for 10 minutes. As a result of the above operations, fluid composition 1 for a crosslinkable relief-forming layer (resin composition for laser engraving) was obtained.

### 2. Preparation of Relief Printing Plate Precursor for Laser Engraving

A spacer (frame) having a predetermined thickness was placed on a PET substrate, coating solution 1 for a crosslinkable relief-forming layer obtained above was cast gently so that it did not overflow from the spacer (frame), and dried in an oven at 70° C. for 3 hours to provide a relief-forming layer having a thickness of 1.28 mm, thus preparing relief printing plate precursor 1 for laser engraving.

### 3. Preparation of Relief Printing Plate

The relief-forming layer of the precursor thus obtained was heated at 80° C. for 3 hours and further at 100° C. for 3 hours to thus thermally crosslink the relief-forming layer.

The crosslinked relief-forming layer was subjected to engraving by the two types of lasers below.

As a carbon dioxide laser engraving machine, an ML-9100 series high quality CO₂ laser marker (KEYENCE) was used for engraving with laser irradiation. After a protection film was peeled off from the printing plate precursor 1 for laser engraving, a 1 cm square solid portion was raster-engraved using the carbon dioxide laser engraving machine under conditions of an output of 12 W, a head speed of 200 mm/sec, and a pitch setting of 2,400 DPI.

As a semiconductor laser engraving machine, laser recording equipment provided with a fiber-coupled semiconductor laser (FC-LD) SDL-6390 (JDSU, wavelength 915 nm) with a maximum power of 8.0 W was used. A 1 cm square solid portion was raster-engraved using the semiconductor laser engraving machine under conditions of a laser output of 7.5 W, a head speed of 409 mm/sec, and a pitch setting of 2,400 DPI.

The thickness of the relief layer of the relief printing plate was about 1 mm and is shown in Table 1.

Furthermore, when the Shore A hardness of the relief layer was measured by the above-mentioned measurement method, it was found to be 78°. Measurement of Shore A hardness was carried out at 25° C. in the same manner in each of the Examples and Comparative Examples described below.

### Examples 2 to 36 and Comparative Examples 1 and 2

The procedure of Example 1 was repeated except that Components A to G used in Example 1 were the materials shown in Table 1, thus preparing relief printing plate precursors of Examples 2 to 36 and Comparative Examples 1 and 2.

The materials used for preparation of these samples are summarized below. The copolymerization ratio is expressed as mol %.

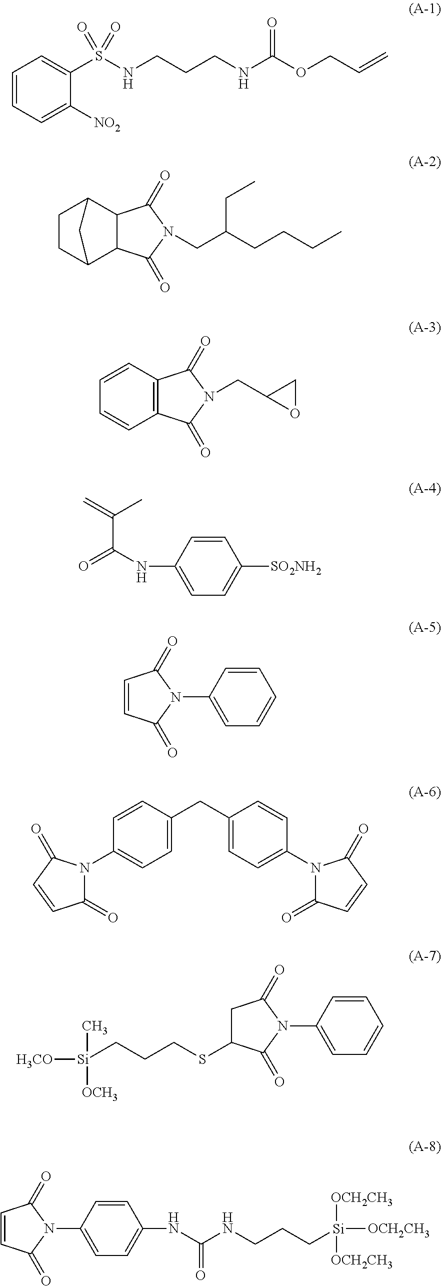

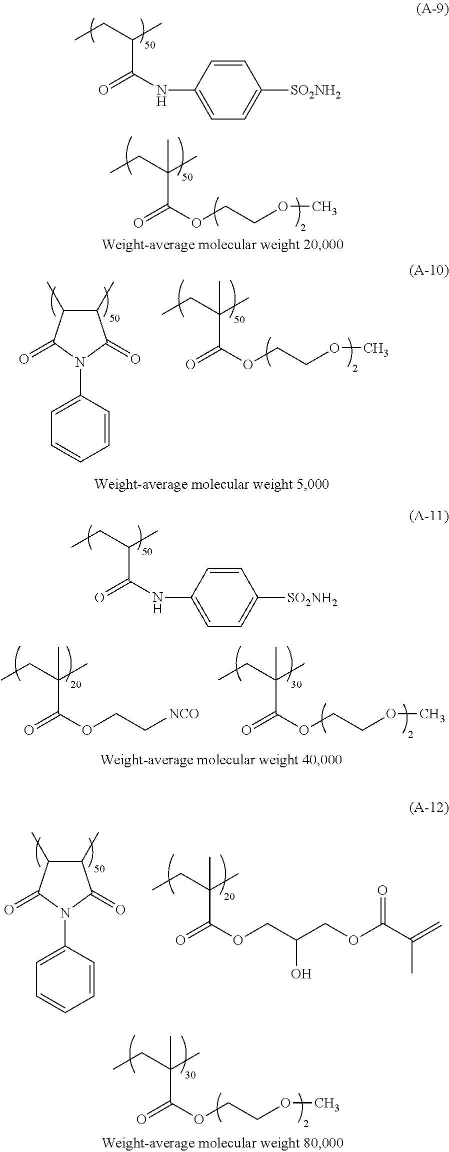

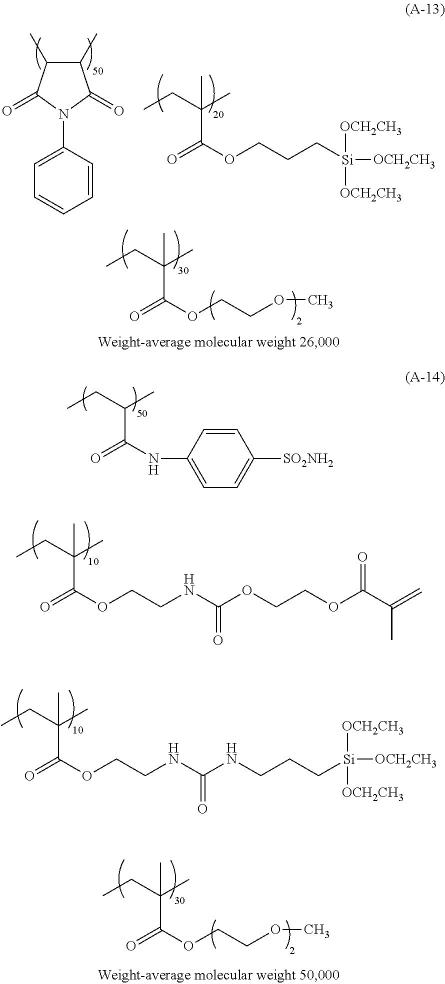

The materials used as Component B in Examples 1 to 36 are shown below.

B-1: polyvinylbutyral (#3000-2, Denki Kagaku Kogyo Kabushiki Kaisha)
B-2: polyamide (Amilan CM4000, Toray)
B-3: butyl methacrylate 70 mol %-methacrylic acid 30 mol % copolymer (synthetic)
B-4: styrene-butadiene rubber (TR2000, JSR)
B-5: styrene-isoprene-styrene block copolymer (SIS5200, JSR)
B-6: hydrogenated styrene-butadiene rubber (DYNARON (registered trademark) 1320P, JSR)
B-7: ester-based polyurethane (Miractran (registered trademark) E-185, Nippon Miractran Co., Ltd.)

The materials used as Component C are shown below.

C-1: glycerol dimethacrylate (Tokyo Chemical Industry Co., Ltd.)
C-2: 1,6-hexanediol diacrylate (Tokyo Chemical Industry Co., Ltd.)

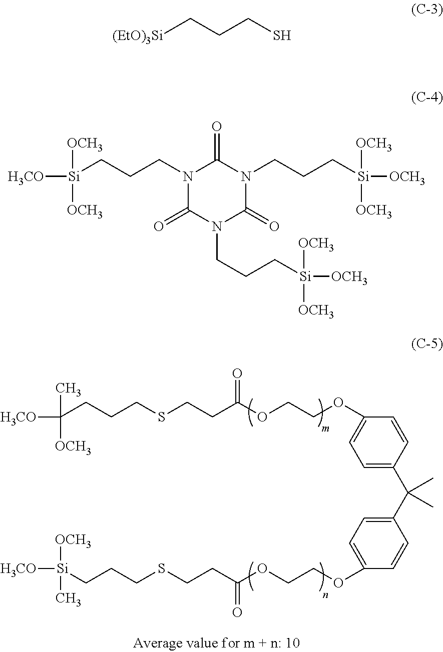

As Component D (polymerization initiator), the radical polymerization initiators below were used.

D-1: dicumyl peroxide (Percumyl D, NOF Corporation)
D-2: t-butylperoxybenzoic acid (Perbutyl Z, NOF Corporation)
D-3: benzoyl peroxide (Nyper BW, NOF Corporation)
D-4: 2,2′-azobis[N-(2-propenyl)-2-methylpropionamide] (VF-096, Wako Pure Chemical Industries, Ltd.)
D-5: 1-hydroxycyclohexyl phenyl ketone (IRGACURE 184, BASF)

Furthermore, with regard to a composition containing an alkoxysilane compound, the compound below was used.

D-6: 1,8-diazabicyclo[5.4.0]undec-7-ene (DBU, San-Apro Ltd.)

As a plasticizer (Component E), the compounds below were used.

E-1: tributyl citrate (Tokyo Chemical Industry Co., Ltd.)
E-2: dioctyl phthalate (Tokyo Chemical Industry Co., Ltd.)

As a fragrance (Component F), those below were used.

F-1: vanillin (Wako Pure Chemical Industries, Ltd.)
F-2: isobornyl acetate (Tokyo Chemical Industry Co., Ltd.)

As a photothermal conversion agent (Component G), those below were used.

### G-1: Ketjen Black (EC600JD, Lion Corporation)

G-2: carbon black (N330, HAF carbon, Tokai Carbon Co., Ltd.)

As a reaction solvent and a coating solvent, γ-butyrolactone was used in all of the Examples and Comparative Examples.

### 2. Preparation of Relief Printing Plate Precursor for Laser Engraving

The procedure of Example 1 was repeated except that the coating solution 1 for a crosslinkable relief-forming layer in Example 1 was changed to coating solutions 2 to 36 for a crosslinkable relief-forming layer and coating solutions C1 and C2 for a comparative crosslinkable relief-forming layer, thus giving relief printing plate precursors 2 to 36 for laser engraving of the Examples and relief printing plate precursors C1 and C2 for laser engraving of the Comparative Examples.

### 3. Preparation of Relief Printing Plate

Relief-forming layers of relief printing plate precursors 2 to 36, C1, and

C2 for laser engraving were subjected to thermal crosslinking and then engraving to form relief layers in the same manner as in Example 1, thereby giving relief printing plates 2 to 36 of the Examples and relief printing plates C1 to C2 of the Comparative Examples.

The thicknesses of the relief layers of these relief printing plates were about 1 mm, as summarized in Table 1.

TABLE 1

| | | | | | | | | | | | | | (Comp. G) | | | |
|---|---|---|---|---|---|---|---|---|---|---|---|---|---|---|---|---|
| | | | | | | | | | | | | | Photo- | | Relief layer | |
| | | | | | (Comp. C) | | (Comp. D) | | | | | | thermal | | | Shore A |
| | | | (Comp. B) | | Polymerizable | | Polymerization | | (Comp. E) | | (Comp. F) | | conversion | | Thickness | hardness |
| Ex. | (Comp. A) | | Binder | | compound | | initiator | | Plasticizer | | Fragrance | | agent | | [mm] | [°] |
| 1 | A-1 | 10 | B-1 | 30 | C-1 | 25 | D-1 | 1 | E-1 | 30 | F-1 | 1 | G-1 | 1 | 1.28 | 78 |
| 2 | A-2 | 10 | B-1 | 30 | C-1 | 25 | D-1 | 1 | E-1 | 30 | F-1 | 1 | G-1 | 1 | 1.28 | 79 |
| 3 | A-3 | 10 | B-1 | 30 | C-1 | 25 | D-1 | 1 | E-1 | 30 | F-1 | 1 | G-1 | 1 | 1.22 | 88 |
| 4 | A-4 | 10 | B-1 | 30 | C-1 | 25 | D-1 | 1 | E-1 | 30 | F-1 | 1 | G-1 | 1 | 1.28 | 72 |
| 5 | A-5 | 10 | B-1 | 30 | C-1 | 25 | D-1 | 1 | E-1 | 30 | F-1 | 1 | G-1 | 1 | 1.11 | 80 |
| 6 | A-6 | 10 | B-1 | 30 | C-1 | 25 | D-1 | 1 | E-1 | 30 | F-1 | 1 | G-1 | 1 | 1.07 | 81 |
| 7 | A-7 | 10 | B-1 | 30 | C-1 | 25 | D-1 | 1 | E-1 | 30 | F-1 | 1 | G-1 | 1 | 1.23 | 75 |
| 8 | A-8 | 10 | B-1 | 30 | C-1 | 25 | D-1 | 1 | E-1 | 30 | F-1 | 1 | G-1 | 1 | 1.30 | 76 |
| 9 | A-9 | 10 | B-1 | 30 | C-1 | 25 | D-1 | 1 | E-1 | 30 | F-1 | 1 | G-1 | 1 | 1.22 | 82 |
| 10 | A-10 | 10 | B-1 | 30 | C-1 | 25 | D-1 | 1 | E-1 | 30 | F-1 | 1 | G-1 | 1 | 1.16 | 80 |
| 11 | A-11 | 10 | B-1 | 30 | C-1 | 25 | D-1 | 1 | E-1 | 30 | F-1 | 1 | G-1 | 1 | 1.21 | 79 |
| 12 | A-12 | 10 | B-1 | 30 | C-1 | 25 | D-1 | 1 | E-1 | 30 | F-1 | 1 | G-1 | 1 | 1.11 | 81 |
| 13 | A-13 | 10 | B-1 | 30 | C-1 | 25 | D-1 | 1 | E-1 | 30 | F-1 | 1 | G-1 | 1 | 1.18 | 85 |
| 14 | A-14 | 10 | B-1 | 30 | C-1 | 25 | D-1 | 1 | E-1 | 30 | F-1 | 1 | G-1 | 1 | 1.12 | 83 |
| 15 | A-13 | 60 | — | 0 | — | 0 | — | 0 | E-1 | 40 | — | 0 | — | 0 | 1.18 | 89 |
| 16 | A-7 | 20 | B-1 | 40 | — | 0 | — | 0 | E-1 | 40 | — | 0 | — | 0 | 1.32 | 75 |
| 17 | A-14 | 15 | B-1 | 40 | C-3 | 5 | D-6 | 1 | E-1 | 40 | — | 0 | — | 0 | 1.28 | 78 |
| 18 | A-14 | 40 | — | 0 | C-5 | 20 | D-6 | 1 | E-1 | 40 | — | 0 | — | 0 | 1.22 | 74 |
| 19 | A-9 | 10 | B-2 | 30 | C-1 | 25 | D-1 | 1 | E-1 | 30 | F-1 | 1 | G-1 | 1 | 1.23 | 74 |
| 20 | A-9 | 10 | B-3 | 30 | C-1 | 25 | D-1 | 1 | E-1 | 30 | F-1 | 1 | G-1 | 1 | 1.20 | 75 |
| 21 | A-9 | 10 | B-4 | 50 | C-1 | 30 | D-1 | 1 | — | 0 | F-1 | 1 | G-1 | 1 | 1.19 | 73 |
| 22 | A-9 | 10 | B-5 | 50 | C-1 | 30 | D-1 | 1 | — | 0 | F-1 | 1 | G-1 | 1 | 1.20 | 78 |
| 23 | A-9 | 10 | B-6 | 50 | C-1 | 30 | D-1 | 1 | — | 0 | F-1 | 1 | G-1 | 1 | 1.12 | 71 |
| 24 | A-9 | 10 | B-7 | 50 | C-1 | 30 | D-1 | 1 | — | 0 | F-1 | 1 | G-1 | 1 | 1.15 | 76 |
| 25 | A-9 | 10 | B-1 | 30 | C-2 | 25 | D-1 | 1 | E-1 | 30 | F-1 | 1 | G-1 | 1 | 1.31 | 77 |
| 26 | A-13 | 10 | B-1 | 30 | C-3 | 25 | D-6 | 1 | E-1 | 30 | F-1 | 1 | G-1 | 1 | 1.22 | 73 |
| 27 | A-13 | 10 | B-1 | 30 | C-4 | 25 | D-6 | 1 | E-1 | 30 | F-1 | 1 | G-1 | 1 | 1.28 | 87 |
| 28 | A-13 | 10 | B-1 | 30 | C-5 | 25 | D-6 | 1 | E-1 | 30 | F-1 | 1 | G-1 | 1 | 1.33 | 80 |
| 29 | A-9 | 10 | B-1 | 30 | C-1 | 25 | D-2 | 1 | E-1 | 30 | F-1 | 1 | G-1 | 1 | 1.19 | 77 |
| 30 | A-9 | 10 | B-1 | 30 | C-1 | 25 | D-3 | 1 | E-1 | 30 | F-1 | 1 | G-1 | 1 | 1.09 | 78 |
| 31 | A-9 | 10 | B-1 | 30 | C-1 | 25 | D-4 | 1 | E-1 | 30 | F-1 | 1 | G-1 | 1 | 1.25 | 87 |
| 32 | A-9 | 10 | B-1 | 30 | C-1 | 25 | D-5 | 1 | E-1 | 30 | F-1 | 1 | — | 0 | 1.30 | 71 |
| 33 | A-9 | 10 | B-1 | 30 | C-1 | 25 | D-1 | 1 | E-2 | 30 | F-1 | 1 | G-1 | 1 | 1.25 | 68 |
| 34 | A-9 | 10 | B-1 | 30 | C-1 | 25 | D-1 | 1 | E-1 | 30 | — | 0 | G-1 | 1 | 1.28 | 73 |
| 35 | A-9 | 10 | B-1 | 30 | C-1 | 25 | D-1 | 1 | E-1 | 30 | F-2 | 1 | G-1 | 1 | 1.22 | 72 |
| 36 | A-9 | 10 | B-1 | 30 | C-1 | 25 | D-1 | 1 | E-1 | 30 | F-1 | 1 | G-2 | 1 | 1.28 | 72 |
| C1 | — | 0 | B-1 | 30 | C-1 | 25 | D-1 | 1 | E-1 | 30 | F-1 | 1 | G-1 | 1 | 1.22 | 75 |
| C2 | — | 0 | B-4 | 50 | C-1 | 30 | D-1 | 1 | — | 0 | F-1 | 1 | G-1 | 1 | 1.19 | 85 |

Note 1:
C1 and C2 denote Comparative Example 1 and Comparative Example 2 respectively
Note 2:
In all of the Examples and Comparative Examples, 20 parts by weight of γ-BL (γ-butyrolactone) was used as a solvent.

### 4. Evaluation of Relief Printing Plate

The performance of a relief printing plate was evaluated in terms of the items below, and the results are summarized in Table 2.

### (1) Evaluation of UV Ink Resistance

A plate was immersed in a UV ink (UV flexographic 500 Indigo, T & K Toka Corporation). After it was left at room temperature for 24 hours, it was taken out, the ink attached to the surface was wiped, the plate was weighed, and the percentage weight loss was calculated using the weight prior to soaking. One with a percentage weight loss of less than 1% was evaluated as Excellent, one with a percentage weight loss of at least 1% but less than 2% as good, one with a percentage weight loss of at least 2% but less than 5% as fair, and one with a percentage weight loss of at least 5% as poor.

### (2) Evaluation of Aqueous Ink Resistance

A plate was immersed in an aqueous ink (Hydric FCG, Dainichiseika Color & Chemicals Mfg. Co., Ltd.). After it was left at room temperature for 24 hours, it was taken out, the ink attached to the surface was wiped, the plate was dried at 120° C. for 1 hour and weighed, and the percentage weight loss was calculated using the weight prior to soaking. One with a percentage weight loss of less than 1% was evaluated as Excellent, one with a percentage weight loss of at least 1% but less than 2% as good, one with a percentage weight loss of at least 2% but less than 5% as fair, and one with a percentage weight loss of at least 5% as poor.

### (3) Evaluation of Ink Washing Agent Resistance

A plate was immersed in an ink washing agent (HEAVY DUTY, FLEXOCLEAN). After it was left at room temperature for 24 hours, it was taken out, the ink washing agent attached to the surface was wiped, the plate was dried at 120° C. for 1 hour and weighed, and the percentage weight loss was calculated using the weight prior to soaking. One with a percentage weight loss of less than 1% was evaluated as Excellent, one with a percentage weight loss of at least 1% but less than 2% as good, one with a percentage weight loss of at least 2% but less than 5% as fair, and one with a percentage weight loss of at least 5% as poor.

TABLE 2

| | | | | Ink washing |
|---|---|---|---|---|
| | | UV ink | Aqueous ink | agent |
| Example | Laser for engraving | resistance | resistance | resistance |
| 1 | Semiconductor laser | Fair | Fair | Fair |
| 2 | Semiconductor laser | Fair | Fair | Fair |
| 3 | Semiconductor laser | Fair | Fair | Fair |
| 4 | Semiconductor laser | Fair | Good | Fair |
| 5 | Semiconductor laser | Fair | Good | Fair |
| 6 | Semiconductor laser | Fair | Good | Good |
| 7 | Semiconductor laser | Good | Good | Fair |
| 8 | Semiconductor laser | Good | Good | Fair |
| 9 | Semiconductor laser | Good | Good | Good |
| 10 | Semiconductor laser | Good | Good | Good |
| 11 | Semiconductor laser | Good | Excellent | Good |
| 12 | Semiconductor laser | Good | Excellent | Excellent |
| 13 | Semiconductor laser | Excellent | Excellent | Good |
| 14 | Semiconductor laser | Excellent | Excellent | Good |
| 15 | Carbon dioxide laser | Fair | Fair | Fair |
| 16 | Carbon dioxide laser | Good | Good | Good |
| 17 | Carbon dioxide laser | Good | Good | Good |
| 18 | Carbon dioxide laser | Good | Good | Good |
| 19 | Carbon dioxide laser | Good | Good | Good |
| 20 | Carbon dioxide laser | Good | Good | Good |
| 21 | Carbon dioxide laser | Fair | Good | Fair |
| 22 | Semiconductor laser | Fair | Good | Fair |
| 23 | Semiconductor laser | Fair | Good | Fair |
| 24 | Semiconductor laser | Fair | Good | Fair |
| 25 | Semiconductor laser | Fair | Good | Fair |
| 26 | Semiconductor laser | Fair | Good | Fair |
| 27 | Semiconductor laser | Good | Good | Fair |
| 28 | Semiconductor laser | Good | Good | Good |
| 29 | Semiconductor laser | Good | Good | Good |
| 30 | Semiconductor laser | Good | Good | Good |
| 31 | Semiconductor laser | Good | Good | Good |
| 32 | Carbon dioxide laser | Good | Good | Good |
| 33 | Semiconductor laser | Good | Good | Good |
| 34 | Semiconductor laser | Good | Good | Good |
| 35 | Semiconductor laser | Good | Good | Good |
| 36 | Semiconductor laser | Good | Good | Good |
| C1 | Semiconductor laser | Poor | Poor | Poor |
| C2 | Semiconductor laser | Poor | Fair | Fair |

Note 1:
C1 and C2 denote Comparative Example 1 and Comparative Example 2, respectively.

1. A resin composition for laser engraving, comprising:
(Component A) a low molecular weight compound containing at least one type of polymerizable group selected from the group consisting of an ethylenically unsaturated group, an epoxy group, an oxetanyl group, a hydrolyzable silyl group, and a silanol group and further containing a sulfonamide group,
a low molecular weight compound containing a residue selected from the group consisting of a maleimide group, a succinimide group, and a phthalimide group, or
a polymer compound containing a constituent unit derived from a compound containing a maleimide group or a constituent unit containing a sulfonamide group.

2. The resin composition for laser engraving according to claim 1, wherein the polymer compound contains an ethylenically unsaturated group or a hydrolyzable silyl group.

3. The resin composition for laser engraving according to claim 1, wherein it further comprises (Component B) a binder polymer.

4. The resin composition for laser engraving according to claim 1, wherein it further comprises (Component C) a polymerizable compound.

5. The resin composition for laser engraving according to claim 1, wherein it further comprises (Component D) a polymerization initiator.

6. The resin composition for laser engraving according to claim 1, wherein it further comprises (Component E) a plasticizer.

7. The resin composition for laser engraving according to claims 1, wherein it further comprises (Component F) a fragrance.

8. The resin composition for laser engraving according to claim 1, wherein it further comprises (Component G) a photothermal conversion agent that can absorb light having a wavelength of 700 to 1,300 nm.

9. A relief printing plate precursor for laser engraving comprising above a support a relief-forming layer comprising the resin composition for laser engraving according to claim 1.

10. A relief printing plate precursor for laser engraving comprising above a support a relief-forming layer formed by crosslinking a relief-forming layer comprising the resin composition for laser engraving according to claim 1 by light and/or heat.

11. A process for producing a relief printing plate precursor for laser engraving, comprising:
a layer formation step of forming a relief-forming layer from the resin composition for laser engraving according to claim 1, and
a crosslinking step of forming a crosslinked relief-forming layer by crosslinking the relief-forming layer by light and/or heat.

12. The process for producing a relief printing plate precursor according to claim 11, wherein the crosslinking step is a step of crosslinking the relief-forming layer by heat.

13. A process for making a relief printing plate, comprising:
a relief layer formation step of forming a relief layer by laser-engraving a relief-forming layer of a relief printing plate precursor for laser engraving produced by the production process according to claim 11.

14. The process for making a relief printing plate according to claim 13, wherein the relief-forming layer has a thickness of at least 0.05 mm but no greater than 10 mm.

15. The process for making a relief printing plate according to claim 13, wherein the relief-forming layer has a Shore A hardness of at least 50° but no greater than 90°.
